(19) United States
(12) Patent Application Publication
(10) Pub. No.: US 2013/0280269 A1
(43) Pub. Date: Oct. 24, 2013

(54) Manipulation of HSP70 and IRE1Alpha Protein Interactions

(71) Applicants: Afshin Samali, Co. Galway (IE); Sanjeev Gupta, Co. Galway (IE); Aysawaria Deepti, Kerala Kochi (IN)

(72) Inventors: Afshin Samali, Co. Galway (IE); Sanjeev Gupta, Co. Galway (IE); Aysawaria Deepti, Kerala Kochi (IN)

(73) Assignee: National University of Ireland, Galway (IE)

(21) Appl. No.: 13/702,649

(22) PCT Filed: Jun. 8, 2011
(86) PCT No.: PCT/IB11/52499

§ 371 Date: May 2, 2013

Related U.S. Application Data
(60) Provisional application No. 61/352,498, filed on Jun. 8, 2010.

(51) Int. Cl. C12N 9/12

(52) U.S. Cl. CPC C12N 9/12
USPC 424/146.1; 435/455; 435/375; 435/69.1; 435/7.21; 424/152.1; 514/44 R

(57) ABSTRACT

The present invention relates to methods that manipulate the level of interaction between the proteins Hsp70 and IRE1α. In particular, the invention relates to methods of manipulating this interaction in order to increase protein yield, and to methods of manipulating this interaction in order to treat diseases associated with abnormal apoptotic activity, including cancer and autoimmune diseases.

### FIELD OF THE INVENTION

The present invention relates to methods that manipulate the level of interaction between the proteins Hsp70 and IRE1α. In particular, the invention relates to methods of manipulating this interaction in order to increase protein yield, and to methods of manipulating this interaction in order to treat diseases associated with abnormal apoptotic activity, including cancer and autoimmune diseases.

### BACKGROUND

The human HSP70 family consists of at least 12 members [1,2]. Of these, the two best studied members are the constitutive or cognate Hsp70 (Hsc70) and its inducible form (Hsp72). Hsc70 is constitutively and ubiquitously expressed in tissues and has basic and essential functions as a molecular chaperone in the folding of proteins [1,2] under normal cellular conditions. Hsp72 is expressed at low levels under normal conditions and its expression is induced upon exposure to environmental stresses such as heat shock, exposure to heavy metals, hypoxia, anoxia and ischemia [1,2].

Physiological or pathological processes that disrupt protein folding in the endoplasmic reticulum lead to ER stress and trigger a set of signalling pathways termed the Unfolded Protein Response (UPR) [16]. This complex cellular response transmits information about the protein-folding status of the ER lumen to the cytosol and nucleus of the cell, resulting in an increase in protein-folding capacity [17,18]. If these mechanisms of cellular adaptation are unable to alleviate the cellular stress [19] the cell will undergo apoptosis, or programmed cell death.

Hsp72 has strong cytoprotective effects and functions as a molecular chaperone in protein folding, transport, and degradation during periods of cellular stress. The cytoprotective effect of Hsp72 is related to its ability to inhibit apoptosis [3,4], which appears to occur by several distinct mechanisms [3,5,6]. For example, Hsp72 prevents the formation of an active apoptosome [7,8], inhibits the release of cytochrome c from the mitochondria [9,10,11] and suppresses JNK, a stress-activated protein kinase [12]. In addition, overexpression of Hsp70 in CHO cells has previously been used as a mechanism for extending the viability of cells in culture [67]. However, to date there has been no indication of the point at which Hsp72 interacts with the apoptotic pathway in order to facilitate these distinct apoptotic mechanisms.

Here, the inventors have surprisingly identified a previously unknown interaction between Hsp72 and inositol requiring enzyme 1 (IRE1α), a transmembrane sensor of ER stress. The inventors have shown that this interaction is responsible for regulating the UPR, and have uncovered novel methods of manipulating this interaction to increase protein production in a cellular system and to treat disorders associated with aberrant apoptosis.

### DETAILED DESCRIPTION OF PREFERRED EMBODIMENTS

The inventors have surprisingly discovered that Hsp72 interacts with IRE1α during the UPR. This interaction implicates Hsp72 in the regulation of IRE1α signalling by modulating the UPRosome [24,25], a complex protein platform that operates at the ER membrane to control IRE1 activity. The interaction between Hsp70 and IRE1α is implicated in controlling each of the UPR pathways which involve IRE1α. The identification of this interaction has multiple applications, both in the field of protein production and in the field of therapy.

In one aspect the invention provides a method of generating a cell for protein production, the method comprising a step of manipulating the cell to increase the level of interaction between Hsp70 and IRE1α, such that the level of unconventionally-spliced XBP1 (XBP1s) mRNA rises and/or the level of JNK or the level of activation of JNK is reduced.

In another aspect the invention provides a method of treating disease, the method comprising a step of modulating the level of interaction between Hsp70 and IRE1α. As a result, the level of unconventionally-spliced XBP1 (XBP1s) mRNA and/or the level of JNK or the level of activation of JNK may be altered.

### The Unfolded Protein Response (UPR)

The Unfolded Protein Response (UPR) is induced in response to any stimuli that causes disruption to ER homeostasis. Such stimuli include agents that cause changes in ER calcium concentration (thapsigargin, calcium ionophore), the disruption of glycosylation (using tunicamycin), the inhibition of ER to Golgi transport (e.g. using brefeldin A), conditions that increase ER protein load (e.g. viral infection), pathological conditions where protein load in the ER increases (e.g. viral infection, accumulation of mutated proteins) and metabolic stress (e.g. exposure to anoxia and ischemia). The basic pathway of the UPR is shown in FIG. 11. The three major transmembrane sensors of ER stress in metazoans are inositol requiring enzyme 1 (IRE1α; also referred to as endoplasmic reticulum-to-nucleus signalling 1 (ERN1)), double-stranded RNA-activated protein kinase (PKR)-like ER kinase (PERK, also referred to as pancreatic eukaryotic initiation factor 2α kinase (PEK) and EIF2AK3), and activating transcription factor 6 (ATF6) [17,18].

IRE1α, the prototype ER stress sensor, is evolutionarily conserved from yeast to humans. IRE1α is a Ser/Thr protein kinase and endoribonuclease that has several functions within the UPR which are believed to operate at different stages of an ER stress response. These functions are depicted in FIG. 14.

IRE1α is activated following oligomerization of the ER luminal domain in response to ER stress. This leads to trans-autophosphorylation, which activates the endoribonculease activity of the protein.

Once activated, IRE1α may initiate the unconventional splicing of the X-box binding protein (XBP1) mRNA [20]. This unconventional splicing includes the excision of a 26 nucleotide long intron of unspliced XBP1 mRNA (XBP1u), which causes a frame shift to occur and a longer version of the XBP1 mRNA (SEQ ID NO: 3) to be produced. Unconventionally spliced XBP1 (XBP1s) mRNA (SEQ ID NO: 3) encodes a highly active transcription factor (SEQ ID NO: 4) which can induce a broad spectrum of UPR-related genes involved in protein folding, protein entry to the ER, ER-associated degradation (ERAD), and protein quality control [21].

XBP1s regulates several UPR target genes including ER chaperones (Grp78, ERdj4, ERdj5, HEDJ, Grp58, and PDIP5), ERAD components (EDEM, HERP, and p58^{1PK}), transcription factors (CHOP and XBP1) and other proteins related to the secretory pathway [23]. This upregulation of genes involved in the UPR leads to a reduction in apoptosis, which is thought to occur at an early stage of the ER stress response.

IRE1α is also involved in the degradation of many mRNAs encoding secretory proteins [82,83]. This pathway has been named Regulated IRE1α-dependent mRNA decay (RIDD; [82]), and is independent of XBP1 splicing. It is thought that this IRE1α pathway predominates at a later stage of the UPR, and functions to reduce the protein folding burden on the ER whilst the levels of stress are brought under control.

A third function of activated IRE1α is in activating c-Jun N-terminal kinases ((JNK), also know as Mitogen Activated Protein Kinase (MAPK)), Extracellular signal-Regulated Kinase (ERK) and nuclear factor kappa-light-chain-enhancer of activated B cells (NFκB). This function is also independent of XBP1 and is achieved by the kinase domain of activated IRE1α, which recruits the TNF receptor-associated factor 2 (TRAF2) adaptor protein [79]. Recruitment of TRAF2 leads to the subsequent recruitment of further accessory proteins including Apoptosis signal-regulating kinase 1 (ASK1) and IκB kinase (IKK) which aid in the recruitment and activation by phosphorylation of the proapoptotic proteins JNK, ERK and NFκB [80,81]. This IRE1α pathway generally occurs at a late stage in the ER stress response when the cell has exhausted all possibilities for avoiding apoptotic cell death.

The inventors have surprisingly discovered that the interaction between IRE1α and Hsp70 is responsible for controlling all three of these roles of IRE1α. Therefore, by altering the interaction between Hsp70 and IRE1α, the pathways controlled by IRE1α can be altered.

For example, by increasing the interaction between Hsp70 and IRE1α, the amount of XBP1s can be increased. This results in the upregulation of genes involved in the UPR whose expression is controlled by XBP1s, and a reduction in apoptosis.

In some embodiments, XBP1s may only be upregulated in response to conditions of cellular stress. Conditions of cellular stress include changes in ER calcium concentration (thapsigargin, calcium ionophore), the disruption of glycosylation (using tunicamycin), the inhibition of ER to Golgi transport (e.g. using brefeldin A), conditions that increase ER protein load (e.g. viral infection), pathological conditions where protein load in the ER increases (e.g. viral infection, accumulation of mutated proteins) and metabolic stress (e.g. exposure to anoxia and ischemia).

In other embodiments, by disrupting the interaction between Hsp70 and IRE1α, the level of JNK activation can be decreased. Since activated JNK leads to enhancement of a pro-apoptotic signalling pathway, disrupting the interaction between Hsp70 and IRE1α, leads to a reduction in apoptosis.

### IRE1α

As discussed above, IRE1α (SEQ ID NO: 6) is a Ser/Thr protein kinase and endoribonuclease that, in response to cellular stress, initiates the unconventional splicing of XBP1 to form the highly active transcription factor XBP1s.

Included within the definition of IRE1α used herein are proteins having 50%, 60%, 70%, 80%, 85%, 90%, 95%, 96%, 97%, 98%, 99% or more sequence identity to SEQ ID NO: 6.

The definition of IRE1α used herein also encompassed fragments of SEQ ID NO: 6. Such fragments may be 100, 200, 300, 400, 500, 600, 700, 800, 900, 950, 960 or more amino acids in length. Also included within the definition of fragments are amino acid sequences which correspond to SEQ ID NO: 6 and have a truncation at the N-terminus and/or the C-terminus. The truncation may be 1, 2, 3, 4, 5, 6, 7, 8, 9, 10, 20, 30, 40, 50, 60, 70, 80, 90, 100, 150, 200 or more amino acids in length. The truncation may be an internal deletion with the same characteristics.

The term IRE1α also encompasses proteins comprising the amino acid sequence of SEQ ID NO: 6 or fragments or truncates thereof.

Fusion proteins comprising the amino acid sequence of SEQ ID NO: 6, or fragments or truncates thereof and a heterologous fusion partner are also contemplated. The fusion partner may be a viral fusion partner. Examples of suitable fusion partners include the HIV-1 TAT protein, the herpes simplex virus 1 (HSV-1) DNA-binding protein VP22, the Drosophila Antennapedia (Antp) homeotic transcription factor, albumin and an Fc region.

### Hsp70

As discussed above, the HSP70 family consists of at least 12 members which function as protein chaperones. [1,2]. Within the scope of the present invention, the Hsp70 can be Hsp70 (SEQ ID NO: 10), Hsp70 (SEQ ID NO: 12), Hsp70-2 (SEQ ID NO: 14), Hsp70-4 (SEQ ID NO: 16), Hsp70-4L (SEQ ID NO: 18), Hsp70-6 (SEQ ID NO: 20), Hsp70-7 (SEQ ID NO: 22), Hsp70-9 (SEQ ID NO: 24), Hsp70-12a (SEQ ID NO: 26), or Hsp70-14 (SEQ ID NO: 28).

In one embodiment the Hsp70 may be Hsp72 (SEQ ID NO: 8).

Included within the definition of Hsp70 used herein are proteins having 50%, 60%, 70%, 80%, 85%, 90%, 95%, 96%, 97%, 98%, 99% or more sequence identity to any one of SEQ ID NOs: 8, 10, 12, 14, 16, 18, 20, 22, 24, 26, or 28.

The definition of Hsp70 used herein also encompasses fragments of any one of SEQ ID NOs: 8, 10, 12, 14, 16, 18, 20, 22, 24, 26, or 28. Such fragments may be 100, 200, 300, 400, 500, 600, 700, 800, 850, 860 or more amino acids in length. Also included within the definition of fragments are amino acid sequences which correspond to any one of SEQ ID NOs: 8, 10, 12, 14, 16, 18, 20, 22, 24, 26, or 28 and have a truncation at the N-terminus and/or the C-terminus. The truncation may be 1, 2, 3, 4, 5, 6, 7, 8, 9, 10, 20, 30, 40, 50, 60, 70, 80, 90, 100, 150, 200 or more amino acids in length. The truncation may be an internal deletion with the same characteristics.

The term Hsp70 also encompasses proteins comprising the amino acid sequence of any one of SEQ ID NOs: 8, 10, 12, 14, 16, 18, 20, 22, 24, 26, or 28 or fragments or truncates thereof.

Fusion proteins comprising the amino acid sequence of any one of SEQ ID NOs: 8, 10, 12, 14, 16, 18, 20, 22, 24, 26, or 28, or fragments or truncates thereof and a heterologous fusion partner are also contemplated. The fusion partner may be a viral fusion partner. Examples of suitable fusion partners include the HIV-1 TAT, protein, the herpes simplex virus 1 (HSV-1) DNA-binding protein VP22, the Drosophila Antennapedia (Antp) homeotic transcription factor, albumin and an Fc region.

### XBP1

As discussed above, XPB1 (SEQ ID NO: 2) is a protein positioned downstream of IRE1α in the UPR cascade. Under conditions of cellular stress IRE1α causes the XBP1 mRNA to be unconventionally spliced. A frame shift which occurs following this unconventional splicing produces the protein XBP1s (SEQ ID NO: 4). This is a longer protein (compared to conventionally spliced XBP1, SEQ ID NO: 2) which functions as a highly active transcription factor, and controls the expression of proteins further down the UPR cascade.

Included within the definition of XBP1 used herein are proteins having 50%, 60%, 70%, 80%, 85%, 90%, 95%, 96%, 97%, 98%, 99% or more sequence identity to SEQ ID NO: 4.

The definition of XBP1 used herein also encompasses fragments of SEQ ID NO: 4. Such fragments may be 50, 100, 200, 300, 350, 370 or more amino acids in length. Also included within the definition of fragments are amino acid sequences which correspond to SEQ ID NO: 4 and have a truncation at the N-terminus and/or the C-terminus. The truncation may be 1, 2, 3, 4, 5, 6, 7, 8, 9, 10, 20, 30, 40, 50, 60, 70, 80, 90, 100, 150, 200 or more amino acids in length. The truncation may be an internal deletion with the same characteristics.

The term XBP1 also encompasses proteins comprising the amino acid sequence of SEQ ID NO: 4 or fragments or truncates thereof.

Fusion proteins comprising the amino acid sequence of SEQ ID NO: 4, or fragments or truncates thereof and a heterologous fusion partner are also contemplated. The fusion partner may be a viral fusion partner. Examples of suitable fusion partners include the HIV-1 TAT protein, the herpes simplex virus 1 (HSV-1) DNA-binding protein VP22, the Drosophila Antennapedia (Antp) homeotic transcription factoralbumin and an Fc region.

### JNK

As discussed above, JNK is a protein positioned downstream of IRE1α in the UPR cascade. Under conditions of extreme cellular stress activated IRE1α recruits TRAF2, which in turn recruits JNK and associated accessory proteins such as ASK 1 and IKK. The formation of this complex leads to JNK activation by phosphorylation.

JNK proteins are encoded by three genes known as JNK1 (MAPK8), JNK2 (MAPK9), and JNK3 (MAPK10). Through alternative splicing these three genes generate 13 isoforms of JNK known as JNK1-α1 (SEQ ID NO: 38, also known as MAPK8-α1), JNK1-β1 (SEQ 1N NO: 39, also known as MAPK8β1, JNK1-β2 (SEQ ID NO: 40, also known as MAPK8-β2), JNK1-α2 (SEQ ID NO: 41, also known as MAPK8-α2), (SEQ ID NO: 43, also known as MAPK9-α1), JNK2-α1 (SEQ 1N NO: 44, also known as MAPK9-β1, JNK2-α2 (SEQ ID NO: 45, also known as MAPK9-α2), JNK2-β2 (SEQ ID NO: 46, also known as MAPK9-β2), JNK2-1 (SEQ ID NO: 47, also known as MAPK9-γ), JNK3 isoform 1 (SEQ ID NO: 49, also known as MAPK10 isoform 1), JNK3 isoform 2 (SEQ ID NO: 50, also known as MAPK10 isoform 2), JNK3 isoform 3 (SEQ ID NO: 51, also known as MAPK10 isoform 3) and JNK3 isoform 4 (SEQ ID NO: 52, also known as MAPK10 isoform 4)

Included within the definition of JNK used herein are proteins having 50%, 60%, 70%, 80%, 85%, 90%, 95%, 96%, 97%, 98%, 99% or more sequence identity to any one of SEQ ID NOs: 38, 39, 40, 41, 43, 44, 45, 46, 47, 49, 50, 51 or 52.

The definition of JNK used herein also encompasses fragments of any one of SEQ ID NOs: 38, 39, 40, 41, 43, 44, 45, 46, 47, 49, 50, 51 or 52. Such fragments may be 50, 100, 200, 250 or more amino acids in length. Also included within the definition of fragments are amino acid sequences which correspond to any one of SEQ ID NOs: 38, 39, 40, 41, 43, 44, 45, 46, 47, 49, 50, 51 or 52 and have a truncation at the N-terminus and/or the C-terminus. The truncation may be 1, 2, 3, 4, 5, 6, 7, 8, 9, 10, 20, 30, 40, 50, 60, 70, 80, 90, 100, 150, 200 or more amino acids in length. The truncation may be an internal deletion with the same characteristics.

The term JNK also encompasses proteins comprising the amino acid sequence of any one of SEQ ID NOs: 38, 39, 40, 41, 43, 44, 45, 46, 47, 49, 50, 51 or 52 or fragments or truncates thereof.

Fusion proteins comprising the amino acid sequence of any one of SEQ ID NOs: 38, 39, 40, 41, 43, 44, 45, 46, 47, 49, 50, 51 or 52, or fragments or truncates thereof and a heterologous fusion partner are also contemplated. The fusion partner may be a viral fusion partner. Examples of suitable fusion partners include the HIV-1 TAT protein, the herpes simplex virus 1 (HSV-1) DNA-binding protein VP22, the Drosophila Antennapedia (Antp) homeotic transcription factoralbumin and an Fc region.

Herein, the term “activated JNK” is used to refer to JNK which is capable of functioning to increase apoptotic signalling relative to non-activated JNK. Activated JNK may be phosphorylated at one or more positions.

Interaction of Hsp70 with IRE1α

The inventors have surprisingly discovered that Hsp70 interacts with IRE1α (see examples 4 and 5 and FIG. 5). This interaction increases the non-conventional splicing of XBP1 and results in increased levels of XBP1s (see examples 2 and 3 and FIGS. 3 and 4). This interaction also increases the activation of JNK through the increased recruitment of TRAF2 and other accessory proteins.

The interaction between Hsp70 and IRE1α may be an electrostatic interaction, a Van der Waals interaction, an ionic interaction, a covalent interaction or a stoichiometric interaction. Alternatively, the interaction between Hsp70 and IRE1α may be formed by a combination of these interactive forces.

The areas of Hsp70 and IRE1α which are required for interaction have also been determined (see example 4 and FIG. 5), and have been identified as the ATPase domain of Hsp70 and the cytosolic C-terminal domain of IRE1α. Therefore, in one embodiment the interaction may be formed with the ATPase domain of Hsp70. In another embodiment, the interaction may be formed with the cytosolic C-terminal domain of IRE1α. The interaction of Hsp70 and IRE1α may require the hydrolysis of ATP. ATP hydrolysis may be facilitated by the ATPase domain of Hsp70.

### Cofactors of Hsp70

It is known that a number of cofactors are required for Hsp70 function [78]. Therefore, in order to interact with and/or for the optimal activation of IRE1α, one or more cofactors may be required. In one embodiment, the cofactors may include, but are not limited to, Hsp40, Hop, Bag 1-5, Hip, HspBP1, CHIP, SGT, Hsp110 homologs, Tom70, and TPR1.

### Method of Generating a Cell for Protein Production

Following the discovery that Hsp70 interacts with IRE1α, and that this interaction increases the levels of XBP1s, leading to a reduced apoptotic rate, the inventors discovered that this principle could be used to enhance protein production in a cellular system.

### Generating a Cell for Protein Production Having Increased Levels of XBP1s

In one embodiment the invention provides a method of generating a cell for protein production, the method comprising a step of manipulating the cell to increase the level of interaction between Hsp70 and IRE1α, such that the level of unconventionally-spliced XBP1 (XBP1s) mRNA rises.

By “such that the level of unconventionally-spliced XBP1 (XBP1s) mRNA rises” is meant that the amount of XBP mRNA present in a manipulated cell is more than the amount of XBP1s mRNA present in an un-manipulated cell. The level of XBP mRNA in a manipulated cell may be 2-fold, 3-fold, 4-fold, 5-fold, 6-fold, 7-fold, 8-fold, 9-fold, 10-fold, 15-fold, 20-fold, 50-fold, 100-fold, 200-fold, 500-fold or more greater than the level of XBP1s mRNA in an un-manipulated cell. This definition applies to all embodiments of the invention.

The increase in the level of XBP1s will lead to an increase in the expression of key UPR genes and to a reduction in the rate of apoptosis. This allows the cell to continue growing in cell culture under conditions of cellular stress. In one embodiment the cellular stress experienced during cell culture may be caused by changes in ER calcium concentration (thapsigargin, calcium ionophore), the disruption of glycosylation (using tunicamycin), the inhibition of ER to Golgi transport (e.g. using brefeldin A), conditions that increase ER protein load (e.g. viral infection), pathological conditions where protein load in the ER increases (e.g. viral infection, accumulation of mutated proteins) and metabolic stress (e.g. exposure to anoxia and ischemia).

The reduced apoptotic rate will allow the cells to produce an increased yield of a protein or proteins of interest. In one embodiment the cell may produce 2-fold, 3-fold, 4-fold, 5-fold, 6-fold, 7-fold, 8-fold, 9-fold, 10-fold, 15-fold, 20-fold or more of a protein of interest than a control cell which has not been manipulated.

In one embodiment the increase in protein yield may be due to an increased level of mRNA. The cell may express the protein of interest at a level of 1, 2, 3, 4, 5, 10, 15, 20, 30, 40, 50, 60, 70, 80, 90, 100, 150, 200, 500, 1000, 10,000 or more mRNA copies per cell relative to the expression level of GAPDH mRNA. The normalisation of the expression level relative to GAPDH is a procedure well known to those skilled in the art.

In one embodiment the Hsp70 may be Hsp72. As described above, Hsp72 has the sequence depicted in SEQ ID NO: 8. Within the scope of the invention, Hsp72 may be a homologue, fragment or fusion protein, as described above.

The skilled person will understand that many mechanisms are known in the art through which the level of the newly identified interaction between Hsp70 and IRE1α can be increased.

In one embodiment the level of interaction between Hsp70 and IRE1α can be increased by increasing the level of expression of Hsp70 and/or IRE1α within the cell. This may involve increasing the level of expression of Hsp70, increasing the level of expression of IRE1α or increasing the level of expression of Hsp70 and IRE1α.

The level of expression of Hsp70 and/or IRE1α may be increased by 2-fold, 3-fold, 4-fold, 5-fold, 6-fold, 7-fold, 8-fold, 9-fold, 10-fold, 15-fold, 20-fold or more when compared to a control cell which has not been manipulated.

In one embodiment the increase in protein yield may be due to an increased level of mRNA. The cell may express the protein of interest at a level of 1, 2, 3, 4 5, 10, 15, 20, 30, 40, 50, 60, 70, 80, 90, 100, 150, 200, 500, 1000, 10,000 or more mRNA copies per cell relative to the expression level of GAPDH mRNA. The normalisation of the expression level relative to GAPDH is a procedure well known to those skilled in the art.

In one embodiment, the step of manipulating the cell may include transfecting the cell with a vector containing a nucleic acid encoding Hsp70 or IRE1α. Vectors suitable for use in the method of the invention include plasmids and viruses (including both bacteriophage and eukaryotic viruses), as well as other linear or circular DNA carriers, such as those employing transposable elements or homologous recombination technology. Particularly suitable viral vectors include baculovirus-, lentivirus-, adenovirus- and vaccinia virus-based vectors.

Suitable transformation or transfection techniques are well known in the art [68]. Within the scope of the invention any method of transfection may be used, including but not limited to calcium phosphate coprecipitation, DEAE dextran facilitated transfection, electroporation, microinjection, cationic liposomes and retroviruses. In eukaryotic cells, expression systems may either be transient (e.g. episomal) or permanent (chromosomal integration) according to the needs of the system.

In one embodiment transfection may be carried out using cationic lipids such as Lipofectamine 2000, Fugene, Turbofect etc. according to manufacturer's instructions.

In another embodiment the method may comprise inserting a strong promoter that is transcriptionally linked to the endogenous gene encoding Hsp70 or IRE1α. A strong promoter is defined as a promoter which is capable of driving the rapid and robust expression of a protein. An example of a strong promoter which may be used within the context of the invention is the Cytomegalovirus (CMV) promoter. In some embodiments the strong promoter may be associated with additional enhancer elements.

A strong promoter may be inserted into the chromosome of the cell by any method known in the art. The promoter, and any enhancer elements, may be inserted by a method of homologous recombination or by a method of site-specific, directed recombination. The strong promoter should be inserted so that it is transcriptionally linked to the endogenous gene encoding Hsp70 or IRE1α such that following insertion of the strong promoter into the cell's chromosome, expression of Hsp70 or IRE1α is increased.

In one embodiment, the expression of co-factors of Hsp70 may also be increased by the insertion of a strong promoter that is transcriptionally linked to the endogenous gene encoding the co-factor. The methods for inserting a strong promoter that is transcriptionally linked to the endogenous gene encoding the co-factor may be the same as the methods used for inserting a strong promoter that is transcriptionally linked to the endogenous gene encoding Hsp70 or IRE1α.

XBP1 splicing is thought to occur in the cytoplasm, and the invention therefore also contemplates increasing the nuclear localisation of XBP1s following unconventional splicing, by the methods discussed above [73].

In another embodiment, the interaction between IRE1α and Hsp70 may be increased by administering an antibody or small molecule to the cell which enhances the level of interaction between IRE1α and Hsp70.

In a further embodiment, the apparent level of interaction been IRE1α and Hsp70 may be increased by administering to the cell an antibody or small molecule which binds to IRE1α and mimics the interaction of Hsp70 with IRE1α.

Within these embodiments, the term antibody includes full chain antibodies and antibody fragments. These include antibody heavy chains, antibody light chains, Fc regions, Fab regions and single chain antibodies. Also contemplated is the use of humanised antibodies, wherein CDR sequences and certain framework residues from a non-human antibody are maintained, and the rest of the antibody residues are substituted for human residues. Methods for the production and humanisation of antibodies are well known in the art.

In one embodiment, the antibody may be a monoclonal antibody.

As it has been determined that Hsp70 and IRE1α interact through the ATPase domain of Hsp70 and the cytosolic C-terminal domain of IRE1α, the antibodies or small molecules for use in these embodiments may be directed against these domains.

### Generating a Cell for Protein Production Having Decreased Levels of Activated JNK

The discovery that the interaction between Hsp70 and IRE1α also controls JNK activation can also be used to enhance protein production in a cellular system.

In one embodiment the invention provides a method of generating a cell for protein production, the method comprising a step of manipulating the cell to decrease the level of interaction between Hsp70 and IRE1α, such that the level of activated JNK or the level of JNK activation decreases.

By “such that the level of activated JNK or the level of JNK activation decreases” is meant that the level of activated JNK or the level of JNK activation present in a manipulated cell is more than the level of activated JNK or the level of JNK activation present in an un-manipulated cell. “Activated JNK” is JNK which is in a form in which it has endoribonuclease activity, and may mean that the JNK molecule is in oligomeric form and/or has been phosphorylated. By “level of JNK activation” is meant the proportion of JNK which is in an active form. The level of activated JNK or the level of JNK activation in a manipulated cell may be 2-fold, 3-fold, 4-fold, 5-fold, 6-fold, 7-fold, 8-fold, 9-fold, 10-fold, 15-fold, 20-fold, 50-fold, 100-fold, 200-fold, 500-fold or more greater than the level of XBP1s mRNA in an un-manipulated cell. This definition applies to all embodiments of the invention.

The decrease in the level of activated JNK or the level of JNK activation will lead to a reduction in the rate of apoptosis. This allows the cell to continue growing in cell culture under conditions of cellular stress. In one embodiment the cellular stress experienced during cell culture may be caused by changes in ER calcium concentration (thapsigargin, calcium ionophore), the disruption of glycosylation (using tunicamycin), the inhibition of ER to Golgi transport (e.g. using brefeldin A), conditions that increase ER protein load (e.g. viral infection), pathological conditions where protein load in the ER increases (e.g. viral infection, accumulation of mutated proteins) and metabolic stress (e.g. exposure to anoxia and ischemia).

The skilled person will understand that there are many ways in which the level of interaction between Hsp70 and IRE1α can be reduced. In one embodiment, the level of interaction may be reduced by administering an Hsp70 neutralising agent or an IRE1α neutralising agent to the cell.

The level of Hsp70 and/or IRE1α may be reduced by 2-fold, 3-fold, 4-fold, 5-fold, 6-fold, 7-fold, 8-fold, 9-fold, 10-fold, 15-fold, 20-fold or more when compared to a control cell which has not been manipulated.

In one embodiment the decrease in protein yield may be due to a decreased level of mRNA. The level of Hsp70 and/or IRE1α mRNA may be reduced to a level of 10⁻¹, 10⁻², 10⁻³, 10⁻⁴, 10⁻⁵, 10⁻⁶ or less mRNA copies per cell relative to the expression level of GAPDH mRNA. The normalisation of the expression level relative to GAPDH is a procedure well known to those skilled in the art.

It will be appreciated that both Hsp70 and IRE1α are involved in complicated pathways within cells, and that both of these proteins have many functions. Therefore, neutralising each of the various functions of either or both of these proteins is likely to have negative effects on a cell. At present it is unclear what the precise effects of a complete neutralisation of either of these proteins would be.

In view of the potential negative effects on the cell, the skilled person would understand that the “neutralising agent” referred to herein is an agent which is capable of disrupting the interaction between Hsp70 and IRE1α. Such a neutralising agent does not necessarily disrupt or affect any of the other functions and properties of these proteins.

In certain embodiments the neutralising agent may be an Hsp70 neutralising agent or an IRE1α neutralising agent. By this is meant that the neutralising agent is directed to either Hsp70 or IRE1α, respectively. Through this, the neutralising agent is able to disrupt the interaction between Hsp70 and IRE1α, thus decreasing the level of activated JNK or the level of JNK activation.

The inventors have determined that the ATPase domain of Hsp70 and the cytosolic C-terminal region of IRE1α are both required for the interaction between Hsp70 and IRE1α.

It is therefore considered that the ATPase domain of Hsp70 is the point at which Hsp70 interacts with IRE1α. Accordingly, an Hsp70 neutralising agent may be directed to the ATPase domain of Hsp70. As depicted in FIG. 5B, the ATPase domain of Hsp70 is defined as amino acids 1-383 of the amino acid sequence of Hsp72 (SEQ ID NO: 8). Therefore, an Hsp70 neutralising agent may be directed towards, and may bind to, one or more of amino acid residues 1-383 of Hsp70.

Alternatively, the ATPase domain of Hsp70 may be required for the interaction with IRE1α because ATP hydrolysis is required for the formation of the interaction.

The cytosolic C-terminal region of IRE1α has been identified as the point at which IRE1α interacts with Hsp70. Accordingly, an IRE1α neutralising agent may be directed to the cytosolic C-terminal region of IRE1α. As depicted in FIG. 5A, the cytosolic C-terminal region of IRE1α is defined as amino acids 500-967 of the amino acid sequence of IRE1α (SEQ ID NO: 16). Therefore, an IRE1α neutralising agent may be directed towards, and may bind to one or more of amino acid residues 500-967 of IRE1α.

In one embodiment the Hsp70 neutralising agent may be an anti-Hsp70 antibody. The term antibody includes full chain antibodies and antibody fragments. These include antibody heavy chains, antibody light chains, Fc regions, Fab regions and single chain antibodies. Also contemplated is the use of humanised antibodies, wherein CDR sequences and certain framework residues from a non-human antibody are maintained, and the rest of the antibody residues are substituted for human residues. Methods for the production and humanisation of antibodies are well known in the art.

In one embodiment, the antibody may be a monoclonal antibody.

An Hsp70 neutralising antibody for use according to the invention may be directed against one or more residues from the ATPase domain of Hsp70. An IRE1α neutralising antibody may be directed against the cytosolic C-terminal domain of IRE1α.

In other embodiments, the Hsp70 neutralising agent may be a small molecule inhibitor of Hsp70. These include VER-155008 [74], 17-dimethylaminoethylamino-17-demethoxygeldanamycin (17-DMAG) [75], 2-Phenylethylnesulfonamide (PES) [76,77], 17-alkyal-amino-17-demethoxygeldanamycin (17-AAG) [77].

The Hsp70 neutralising agent or the IRE1α neutralising agent may also be a peptide aptamer, DNA aptamer, RNA aptamer or siRNA molecule.

### Expressed Protein

The protein to be expressed by the cell may be a therapeutic or vaccine protein. In one embodiment such proteins may include insulin and erythropoietin.

In one embodiment the protein to be expressed by the cell may be a secreted protein or a membrane protein such as cytokines, interferons, streptokinase, urokinase etc.

### Cell for Protein Production

The cell for protein production may be a eukaryotic cell. Where the cell is a eukaryotic cell it may be a fungal cell (e.g. yeast; S. cerevisiae, S. Pombe, P. Pastoris), a plant cell or an animal cell. Where the cell is an animal cell it may be a mammalian cell or an insect cell. Where the cell is a mammalian cell it may, for example, be a PC12 cell, a CHO cell, an NS0 cell, a BHK cell, a human retinal cell or an HEK-293 cell.

In one embodiment the method of the invention may comprise isolating the protein of interest from the cell preparation. Methods of isolation will be well known to a person skilled in the art, and any method of protein isolation may be utilised within the scope of the invention. In particular, the protein may be isolated by immunoprecipitation, immunoelectrophoresis, chromatographic methods, gel electrophoresis, centrifugal methods, or any combination of such methods.

The invention also includes a cell produced according to any of the methods of the invention.

### Methods of Treatment

### Methods of Treatment Requiring an Alteration in the Level of XBP1s

In another aspect the invention provides a method of treating disease, the method comprising a step of modulating the level of interaction between Hsp70 and IRE1α, such that the level of unconventionally-spliced XBP1 (XBP1s) mRNA is altered.

The term “modulating” includes any form of altering the level of interaction between Hsp70 and IRE1α including increasing the level of interaction between the two proteins and decreasing the level of interaction between the two proteins.

Treatment of Diseases Associated with a Decreased Level of Apoptosis

As discussed above, the interaction between Hsp70 and IRE1α causes the unconventional splicing of XBP1 such that the level of unconventionally-spliced XBP1 (XBP1s) mRNA is increased. Therefore, decreasing the level of interaction between Hsp70 and IRE1α, will lead to a reduction in the level of unconventionally-spliced XBP1 (XBP1s) mRNA.

A reduction in the level of XBP1s in a cell will lead to the reduced adaptive response initiated by IRE1/XBP1 branch of the UPR. Since the UPR functions to prevent cellular apoptosis, a reduction in this response will lead to cellular apoptosis. This can be advantageous for the treatment of disorders and diseases associated with a reduced rate of apoptosis. Therefore, the method of this aspect of the invention may be used to treat a disease or disorder associated with a reduced rate of apoptosis.

Cancer and metastatic disorders and diseases are particularly known for having a reduced rate of apoptosis. Following a series of events which trigger a cell to follow a metastatic pathway, the cell can become resistant to apoptosis, leading to tumour formation. This reduced rate of apoptosis may, in part, be due to an increase in the UPR, which allows cells to remain alive even under severely stressed conditions. Therefore, decreasing the interaction between Hsp70 and IRE1α, will lead to a reduction in the level of unconventionally-spliced XBP1 (XBP1s) mRNA, and a reduction in the UPR. This will allow cells to undergo apoptosis and will alleviate cancer and metastatic disorders and diseases.

The centre of a solid tumour is a particularly stressed environment for a cell due to the reduced oxygen levels and the potential for anoxia and hypoxia. In addition, the use of conventional chemotherapeutic drugs such as cisplatin, carboplatin, oxaliplatin, mechlorethamine, cyclophosphamide, chlorambucil, ifosfamide, vinca alkaloids such as vincristine, vinblastine, vinorelbine, or vindesine, taxanes such as taxol or docetaxel, camptothecins, irinotecan, topotecan, amsacrine, etoposide, etoposide phosphate, teniposide, doxorubicin, epirubicin, bleomycin, podophyllotoxin, etoposide, and teniposide can further increase the stressed nature of the environment within a solid tumour. Therefore, a reduction in the UPR by reducing the interaction between Hsp70 and IRE1α will enable cells living within such a stressed environment to proceed down the apoptotic pathway.

In one embodiment the cancer or metastatic disorder or disease may comprise a solid tumor.

In another embodiment the cancer or metastatic disorder or disease may be a carcinoma, a sarcoma, a lymphoma, multiple myeloma or a blastoma.

In another embodiment the cancer may be breast cancer, lung cancer, colon cancer, cervical cancer, prostate cancer, ovarian cancer, leukaemia, or bowel cancer.

The skilled person will understand that there are many ways in which the level of interaction between Hsp70 and IRE1α can be reduced. In one embodiment, the level of interaction may be reduced by administering an Hsp70 neutralising agent or an IRE1α neutralising agent to the affected cell.

The level of Hsp70 and/or IRE1α may be reduced by 2-fold, 3-fold, 4-fold, 5-fold, 6-fold, 7-fold, 8-fold, 9-fold, 10-fold, 15-fold, 20-fold or more when compared to a control subject.

In one embodiment the decrease in protein yield may be due to a decreased level of mRNA. The level of Hsp70 and/or IRE1α mRNA may be reduced to a level of 10⁻¹, 10⁻², 10⁻³, 10⁻⁴, 10⁻⁵, 10⁻⁶ or less mRNA copies per cell relative to the expression level of GAPDH mRNA. The normalisation of the expression level relative to GAPDH is a procedure well known to those skilled in the art.

It will be appreciated that both Hsp70 and IRE1α are involved in complicated pathways within cells, and that both of these proteins have many functions. Therefore, neutralising each of the various functions of either or both of these proteins is likely to have negative effects on a subject. At present it is unclear what the precise effects of a complete neutralisation of either of these proteins would be.

In view of the potential side effects, the skilled person would understand that the “neutralising agent” referred to herein is an agent which is capable of disrupting the interaction between Hsp70 and IRE1α. Such a neutralising agent does not necessarily disrupt or affect any of the other functions and properties of these proteins.

In certain embodiments the neutralising agent may be an Hsp70 neutralising agent or an IRE1α neutralising agent. By this is meant that the neutralising agent is directed to either Hsp70 or IRE1α, respectively. Through this, the neutralising agent is able to disrupt the interaction between Hsp70 and IRE1α.

The inventors have determined that the ATPase domain of Hsp70 and the cytosolic C-terminal region of IRE1α are both required for the interaction between Hsp70 and IRE1α.

It is therefore considered that the ATPase domain of Hsp70 is the point at which Hsp70 interacts with IRE1α. Accordingly, an Hsp70 neutralising agent may be directed to the ATPase domain of Hsp70. As depicted in FIG. 5B, the ATPase domain of Hsp70 is defined as amino acids 1-383 of the amino acid sequence of Hsp72 (SEQ ID NO: 8). Therefore, an Hsp70 neutralising agent may be directed towards, and may bind to one or more of amino acid residues 1-383 of Hsp70.

Alternatively, the ATPase domain of Hsp70 may be required for the interaction with IRE1α because ATP hydrolysis is required for the formation of the interaction.

The cytosolic C-terminal region of IRE1α has been identified as the point at which IRE1α interacts with Hsp70. Accordingly, an IRE1α neutralising agent may be directed to the cytosolic C-terminal region of IRE1α. As depicted in FIG. 5A, the cytosolic C-terminal region of IRE1α is defined as amino acids 500-967 of the amino acid sequence of IRE1α (SEQ ID NO: 16). Therefore, an IRE1α neutralising agent may be directed towards, and may bind to one or more of amino acid residues 500-967 of IRE1α.

In one embodiment the Hsp70 neutralising agent may be an anti-Hsp70 antibody. The term antibody includes full chain antibodies and antibody fragments. These include antibody heavy chains, antibody light chains, Fc regions, Fab regions and single chain antibodies. Also contemplated is the use of humanised antibodies, wherein CDR sequences and certain framework residues from a non-human antibody are maintained, and the rest of the antibody residues are substituted for human residues. Methods for the production and humanisation of antibodies are well known in the art.

In one embodiment, the antibody may be a monoclonal antibody.

An Hsp70 neutralising antibody for use according to the invention may be directed against one or more residues from the ATPase domain of Hsp70. An IRE1α neutralising antibody may be directed against the cytosolic C-terminal domain of IRE1α.

In other embodiments, the Hsp70 neutralising agent may be a small molecule inhibitor of Hsp70. These include VER-155008 [74], 17-dimethylaminoethylamino-17-demethoxygeldanamycin (17-DMAG) [75], 2-Phenylethylnesulfonamide (PES) [76,77], 17-alkyal-amino-17-demethoxygeldanamycin (17-AAG) [77].

The Hsp70 neutralising agent or the IRE1α neutralising agent may also be a peptide aptamer, DNA aptamer, RNA aptamer or siRNA molecule.

In one embodiment, the invention also provides an Hsp70 neutralising agent or an IRE1α neutralising agent for use in the treatment of cancer or a metastatic disease or disorder. The invention also provides the use of an Hsp70 neutralising agent or an IRE1α neutralising agent in the manufacture of a medicament for the treatment of cancer or a metastatic disease or disorder.

Also included within the scope of the invention is a pharmaceutical composition comprising an Hsp70 neutralising agent or an IRE1α neutralising agent as defined above and a pharmaceutically acceptable carrier, excipient, diluent or buffer.

The term “pharmaceutically acceptable carrier” may include liquids such as water, saline, glycerol, ethanol or auxiliary substances such as wetting or emulsifying agents, pH buffering substances and the like. Excipients may enable the pharmaceutical compositions to be formulated into tablets, pills, capsules, liquids, gels, or syrups to aid intake by the subject. A thorough discussion of pharmaceutically acceptable carriers is available in Remington's Pharmaceutical Sciences [69].

In one embodiment, the pharmaceutical composition may include an Hsp70 neutralising agent and an IRE1α neutralising agent. In another embodiment the pharmaceutical composition may include one or more Hsp70 neutralising agents and one or more IRE1α neutralising agents.

The pharmaceutical composition may also include one or more additional therapeutic agents. In particular the pharmaceutical composition may include one or more additional anti-cancer agents such as cisplatin, carboplatin, oxaliplatin, mechlorethamine, cyclophosphamide, chlorambucil, ifosfamide, vinca alkaloids such as vincristine, vinblastine, vinorelbine, or vindesine, taxanes such as taxol or docetaxel, camptothecins, irinotecan, topotecan, amsacrine, etoposide, etoposide phosphate, teniposide, doxorubicin, epirubicin, bleomycin, podophyllotoxin, etoposide, or teniposide.

In a further embodiment the pharmaceutical composition may comprise one or more further pro-apoptotic agents. Such agents include, TRAIL, TRAIL mutants including those described in WO2005/056596, WO2009/077857, WO/2009/066174, Bax, Bid, Bak, or Bad.

As used herein, the term “treatment” is considered to encompass therapy, and can be prophylactic or therapeutic.

The neutralising agents or pharmaceutical compositions described above may be used for the treatment of disease in any animal. The animal may be a mammal such as a cow, pig, camel, goat, sheep, cat, dog or rabbit. In one embodiment, the mammal may be a human.

The neutralising agent or pharmaceutical composition may be formulated as a pill, syrup, injectable solution, cream, ointment, suppository, pessary, spray, eye drop or the neutralising agent or pharmaceutical composition may be inserted into or coated onto a medical device useful for the treatment of a patient, such as a stent, stitch, rod or mesh.

The neutralising agent or pharmaceutical composition of the invention may be administered to a patient using one or more of a number of modes of administration which will be known to a person skilled in the art. Such modes of administration may include parenteral injection (e.g. intravenously, subcutaneously, intraperitoneally, intramuscularly, or to the interstitial space of a tissue), or by rectal, oral, vaginal, topical, transdermal, intradermal, intrathecal, intranasal, ocular, aural, pulmonary or other mucosal administration. The precise mode of administration will depend on the disease or condition to be treated.

Treatment Disorders Associated with Increased Apoptosis

An increase in the level of interaction between Hsp70 and IRE1α will lead to an increase in XBPs. This will lead to an increase in the UPR and to a reduction in the apoptotic rate. This can be useful for the treatment of a disorders or diseases associated with an increased rate of apoptosis. Therefore, in another aspect the invention provides a method of treating a disease or disorder associated with an increased rate of apoptosis by increasing the level of interaction between Hsp70 and IRE1α, such that the level of unconventionally-spliced XBP1 mRNA rises.

Autoimmune disorders and diseases, allergies or other hypersensitivity disorders, diabetes, cardiac disease, transplant rejection and graft-versus-host disease, and acute and chronic inflammatory diseases are particularly known for having an increased rate of apoptosis.

Within the scope of the invention the autoimmune disorders include but are not limited to achlorhydra autoimmune chronic active hepatitis, Addison's disease, alopecia greata, amyotrophic lateral sclerosis (ALS, Lou Gehrig's Disease), ankylosing spondylitis, anti-GBM nephritis or anti-TBM nephritis, antiphospholipid syndrome, aplastic anemia, arthritis, asthma, atopic allergy, atopic dermatitis, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Balo disease, Behcet's disease, Berger's disease (IgA Nephropathy), bullous pemphigoid, cardiomyopathy, celiac disease, celiac sprue dermatitis, chronic fatigue immune deficiency syndrome (CFIDS), chronic fatigue immune dysfunction syndrome (CFIDS), chronic inflammatory demyelinating polyneuropathy, Churg Strauss syndrome, cicatricial pemphigoid, Cogan's syndrome, cold agglutunin disease, colitis, cranial arteritis, CREST syndrome, Crohn's disease, Cushing's syndrome, Dego's disease, dermatitis, dermatomyositis, dermatomyositis—juvenile, Devic's disease, type 1 diabetes, discoid lupus, Dowling-Dego's disease, Dressler's syndrome, eosinophilic fasciitis, epidermolysis bullosa acquisita, essential mixed cryoglobulinemia, Evan's syndrome, fibromyalgia, fibromyositis, fibrosing alveolitis, gastritis, giant cell artertis, glomerulonephritis, Goodpasture's disease, Grave's disease, Guillian-Barre syndrome, Hashimoto's thyroiditis, hemolytic anemia, Henoch-Schonlein purpura, hepatitis, Hughes syndrome, idiopathic adrenal atrophy, idiopathic pulmonary fibrosis, idiopathic thrombocytopenia purpura (ITP), IgA nephropathy, inflammatory demylinating polyneuropathy, insulin dependent diabetes (Type I), irritable bowel syndrome, juvenile arthritis, Kawasaki's disease, lichen planus, Lou Gehrig's disease, lupoid hepatitis, Lyme disease, Meniere's disease, mixed connective tissue disease, multiple myeloma, multiple sclerosis, myasthenia gravis, myositis, ocular cicatricial pemphigoid, osteoporosis, pars planitis, pemphigus vulgaris, pernicious anemia, polyarteritis nodosa, polychondritis, polyglandular syndromes, polyglandular autoimmune syndromes, polymyalgia rheumatica (PMR), polymyositis, polymyositis and dermatomyositis, primary agammaglobulinemia, primary biliary cirrhois, primary biliary cirrhosis, primary sclerosing cholangitis, psoriasis, Raynaud's phenomenon, Reiter's syndrome, rheumatoid arthritis, sarcoidosis, scleritis, scleroderma, Sjogren's syndrome, sticky blood syndrome, stiff-man syndrome, Still's disease, Sydenham's chorea, systemic lupus erythmatosis (SLE), Takayasu's arteritis, temporal arteritis/giant cell arteritis, ulcerative colitis, uveitis, vasculitis, vitiligo, Wegener's granulomatosis, and Wilson's syndrome.

The allergy or hypersensitivity disorder may be any known allergy or hypersensitivity disorder including type I, type II, type III, or type IV according to the Gell-Coombs classification, and the less commonly defined type V hypersensitivity disorders. Such disorders include but are not limited to atopy, asthma, ertyhroblastosis fetalis, Goodpasture's syndrome, autoimmune hemolytic anemia, serum sickness, Arthus reaction, systemic lupus erythematosus, contact dermatitis, tuberculin skin test, chronic transplant rejection, Graves disease, myasthenia gravis, systemic anaphylaxis, local anaphylaxis, allergic rhinitis, conjunctivitis, gastroenteritis, eczema, blood transfusion reactions, haemolytic disease of the newborn, rheumatoid arthritis, glomerulonephritis, contact dermatitis, atopic dermatitis, tubercular lesions, drug-induced hemolytic anemia, lupus nephritis, aspergillosis, polyarteritis, polymyositis, scleroderma, hypersensitivity pneumonitis, Wegener's granulomastosis, type I diabetes mellitus, urticaria/angioedema, or inflammation of the thyroid. The allergy or hypersensitivity disorder may be associated with infectious diseases including but not limited to tuberculosis, leprosy, blastomycosis, histoplasmosis, toxoplasmosis, leishmaniasis or other infections. Allergies that may be treated include but are not limited to allergic reactions to pollens (e.g. birch tree, ragweed, oil seed rape), food (e.g. nuts, eggs or seafood), drugs (e.g. penicillin or salicylates), insect products (e.g. bee or wasp venom or house dust mites) or animal hair, and man-made products such as latex. Other inflammatory diseases that may be treated include atherosclerosis, Alzheimer's disease, vasculisitis, myositis, encephalitis, reperfusion injury and wound healing, including the inflammatory phase, the process of angiogenesis, fibroplasmia and epithelialisation, and the remodeling phase.

In one embodiment the method may comprise increasing the level of Hsp70 or IRE1α in a patient's cells. In another embodiment the method may comprise increasing the level of both Hsp70 and IRE1α.

The level of Hsp70 and/or IRE1α may be increased by 2-fold, 3-fold, 4-fold, 5-fold, 6-fold, 7-fold, 8-fold, 9-fold, 10-fold, 15-fold, 20-fold or more when compared to a control subject.

In one embodiment the increase in protein yield may be due to an increased level of mRNA. The cell may express the protein of interest at a level of 1, 2, 3, 4, 5, 10, 15, 20, 30, 40, 50, 60, 70, 80, 90, 100, 150, 200, 500, 1000, 10,000 or more mRNA copies per cell relative to the expression level of GAPDH mRNA. The normalisation of the expression level relative to GAPDH is a procedure well known to those skilled in the art.

In one embodiment the level of Hsp70 or IRE1α may be increased by administering an Hsp70 or IRE1α protein to the patient in need of treatment in a therapeutically effective amount.

The Hsp70 or IRE1α protein for administration to a patient may be formulated for administration in any pharmaceutically acceptable form.

If both Hsp70 and IRE1α are to be administered, these may be administered by separate, sequential or simultaneous administration.

In another embodiment the level of Hsp70 or IRE1α may be increased by increasing the level of expression of Hsp70 or IRE1α. This can be achieved by administering a vector encoding Hsp70 or IRE1α in a therapeutically effective amount to a patient in need of treatment. Vectors suitable for use in the method of the invention include plasmids and viruses (including both bacteriophage and eukaryotic viruses), as well as other linear or circular DNA carriers, such as those employing transposable elements or homologous recombination technology; and host cells including attenuated host cells. S. cerevisiae is an example of a suitable host cell. Particularly suitable viral vectors include baculovirus-, lentivirus-, adenovirus- and vaccinia virus-based vectors.

Expression vectors for use in the method of the invention may incorporate the appropriate transcriptional and translational control sequences, for example enhancer elements, promoter-operator regions, termination stop sequences, mRNA stability sequences, start and stop codons or ribosomal binding sites, linked in frame with the nucleic acid molecules of the invention. These control sequences are provided by way of example only, and are not intended to be limited.

In one embodiment, the level of interaction between Hsp70 and IRE1α may be increased by increasing the level of co-factors of Hsp70. Hsp40 is an example of such a co-factor. The level of cofactor may be increased by any of the methods described above in relation to Hsp70, including administering the protein to the subject, administering a vector encoding the protein to a subject and administering a nuclear localised version of the co-factor, or a vector encoding said co-factor, to a subject.

In another embodiment, the interaction between IRE1α and Hsp70 may be increased by administering an antibody or small molecule to the patient which enhances the level of interaction between IRE1α and Hsp70.

In a further embodiment, the apparent level of interaction been IRE1α and Hsp70 may be increased by administering to the patient an antibody or small molecule which binds to IRE1α and mimics the interaction of Hsp70 with IRE1α.

Within these embodiments, the term antibody includes full chain antibodies and antibody fragments. These include antibody heavy chains, antibody light chains, Fc regions, Fab regions and single chain antibodies. Also contemplated is the use of humanised antibodies, wherein CDR sequences and certain framework residues from a non-human antibody are maintained, and the rest of the antibody residues are substituted for human residues. Methods for the production and humanisation of antibodies are well known in the art.

In one embodiment, the antibody may be a monoclonal antibody.

As it has been determined that Hsp70 and IRE1α interact through the ATPase domain of Hsp70 and the cytosolic C-terminal domain of IRE1α, the antibodies or small molecules for use in these embodiments may be directed against these domains.

In certain embodiments the method of the invention may also comprise inducing cellular stress. The skilled person will understand that various types of cellular stress may be induced, including changes in ER calcium concentration (thapsigargin, calcium ionophore), the disruption of glycosylation (using tunicamycin), the inhibition of ER to Golgi transport (e.g. using brefeldin A), conditions that increase ER protein load (e.g. viral infection), pathological conditions where protein load in the ER increases (e.g. viral infection, accumulation of mutated proteins) and metabolic stress (e.g. exposure to anoxia and ischemia). Within the scope of the invention the method may include inducing cellular stress through inducing one or more of these stressed states. Medical conditions in which cellular stress is a feature require no methods for induction of cellular stress and are preferred conditions in which the present invention may be exploited. Such conditions are listed herein.

The invention also includes a pharmaceutical composition comprising the amino acid sequence of Hsp70 or a vector encoding Hsp70, or the amino acid sequence of IRE1α or a vector encoding IRE1α and a pharmaceutically acceptable carrier, excipient, diluent or buffer.

The term “pharmaceutically acceptable carrier” may include liquids such as water, saline, glycerol, ethanol or auxiliary substances such as wetting or emulsifying agents, pH buffering substances and the like. Excipients may enable the pharmaceutical compositions to be formulated into tablets, pills, capsules, liquids, gels, or syrups to aid intake by the subject. A thorough discussion of pharmaceutically acceptable carriers is available in Remington's Pharmaceutical Sciences [69].

In one embodiment the invention includes the Hsp70, IRE1α, or Hsp40 protein, nucleic acid encoding Hsp70, IRE1α, or Hsp40, or pharmaceutical composition described above for use in the treatment of autoimmune disorders and diseases, allergies or other hypersensitivity disorders, diabetes, cardiac disease, transplant rejection and graft-versus-host disease, and acute and chronic inflammatory diseases.

In another embodiment the invention includes the use of the Hsp70, IRE1α, or Hsp40 protein, nucleic acid encoding Hsp70, IRE1α, or Hsp40, or pharmaceutical composition described above in the manufacture of a medicament for the treatment of autoimmune disorders and diseases, allergies or other hypersensitivity disorders, diabetes, cardiac disease, transplant rejection and graft-versus-host disease, and acute and chronic inflammatory diseases.

As used herein, the term “treatment” is considered to encompass therapy, and can be prophylactic or therapeutic.

The Hsp70, IRE1α, or Hsp40 protein, nucleic acid encoding Hsp70, IRE1α, or Hsp40, or pharmaceutical composition described above may be used for the treatment of disease in any animal. The animal may be a mammal such as a cow, pig, camel, goat, sheep, cat, dog or rabbit. In one embodiment, the mammal may be a human.

The Hsp70, IRE1α, or Hsp40 protein, nucleic acid encoding Hsp70, IRE1α, or Hsp40, or pharmaceutical composition may be formulated as a pill, syrup, injectable solution, cream, ointment, suppository, pessary, spray, eye drop or the Hsp70, IRE1α, or Hsp40 protein, nucleic acid encoding Hsp70, IRE1α, or Hsp40 or pharmaceutical composition may be inserted into or coated onto a medical device useful for the treatment of a patient, such as a stent, stitch, rod or mesh.

The Hsp70, IRE1α, or Hsp40 protein, nucleic acid encoding Hsp70, IRE1α, or Hsp40, or pharmaceutical composition of the invention may be administered to a patient using any one or more of a number of modes of administration which will be known to a person skilled in the art. Such modes of administration may include parenteral injection (e.g. intravenously, subcutaneously, intraperitoneally, intramuscularly, or to the interstitial space of a tissue), or by rectal, oral, vaginal, topical, transdermal, intradermal, intrathecal, intranasal, ocular, aural, pulmonary or other mucosal administration. The precise mode of administration will depend on the disease or condition to be treated.

### Methods of Treatment Requiring an Alteration in the Level of Activated JNK

In another aspect the invention provides a method of treating disease, the method comprising a step of modulating the level of interaction between Hsp70 and IRE1α, such that the level or level of activated JNK is altered.

The term “modulating” includes any form of altering the level of interaction between Hsp70 and IRE1α including increasing the level of interaction between the two proteins and decreasing the level of interaction between the two proteins.

Treatment of Diseases Associated with a Decreased Level of Apoptosis

As discussed above, the interaction between Hsp70 and IRE1α causes the activation of JNK. Therefore, increasing the level of interaction between Hsp70 and IRE1α, will lead to an increase in the level of activated JNK or the level of activation of JNK.

An increase in the level of activated JNK or the level of activation of JNK in a cell will lead to increased apoptotic signalling and an increased rate of cellular apoptosis. This can be advantageous for the treatment of disorders and diseases associated with a reduced rate of apoptosis. Therefore, the method of this aspect of the invention may be used to treat a disease or disorder associated with a reduced rate of apoptosis.

Cancer and metastatic disorders and diseases are particularly known for having a reduced rate of apoptosis. Following a series of events which trigger a cell to follow a metastatic pathway, the cell can become resistant to apoptosis, leading to tumour formation. This reduced rate of apoptosis may, in part, be due to an increase in the UPR, which allows cells to remain alive even under severely stressed conditions. Therefore, increasing the interaction between Hsp70 and IRE1α, will lead to an increase in the level of activated JNK, and a relative reduction in the UPR. This will allow cells to undergo apoptosis and will alleviate cancer and metastatic disorders and diseases.

In one embodiment the cancer or metastatic disorder or disease may comprise a solid tumor.

In another embodiment the cancer or metastatic disorder or disease may be a carcinoma, a sarcoma, a lymphoma, multiple myeloma or a blastoma.

In another embodiment the cancer may be breast cancer, lung cancer, colon cancer, cervical cancer, prostate cancer, ovarian cancer, leukaemia, or bowel cancer.

The skilled person will understand that there are many ways in which the level of interaction between Hsp70 and IRE1α can be increased.

In one embodiment the method may comprise increasing the level of Hsp70 or IRE1α in a patient's cells. In another embodiment the method may comprise increasing the level of both Hsp70 and IRE1α.

The level of Hsp70 and/or IRE1α may be increased by 2-fold, 3-fold, 4-fold, 5-fold, 6-fold, 7-fold, 8-fold, 9-fold, 10-fold, 15-fold, 20-fold or more when compared to a control subject.

In one embodiment the increase in protein yield may be due to an increased level of mRNA. The cell may express the protein of interest at a level of 1, 2, 3, 4, 5, 10, 15, 20, 30, 40, 50, 60, 70, 80, 90, 100, 150, 200, 500, 1000, 10,000 or more mRNA copies per cell relative to the expression level of GAPDH mRNA. The normalisation of the expression level relative to GAPDH is a procedure well known to those skilled in the art.

In one embodiment the level of Hsp70 or IRE1α may be increased by administering an Hsp70 or IRE1α protein to the patient in need of treatment in a therapeutically effective amount.

The Hsp70 or IRE1α protein for administration to a patient may be formulated for administration in any pharmaceutically acceptable form.

If both Hsp70 and IRE1α are to be administered, these may be administered by separate, sequential or simultaneous administration.

In another embodiment the level of Hsp70 or IRE1α may be increased by increasing the level of expression of Hsp70 or IRE1α. This can be achieved by administering a vector encoding Hsp70 or IRE1α in a therapeutically effective amount to a patient in need of treatment. Vectors suitable for use in the method of the invention include plasmids and viruses (including both bacteriophage and eukaryotic viruses), as well as other linear or circular DNA carriers, such as those employing transposable elements or homologous recombination technology; and host cells including attenuated host cells. S. cerevisiae is an example of a suitable host cell. Particularly suitable viral vectors include baculovirus-, lentivirus-, adenovirus- and vaccinia virus-based vectors.

Expression vectors for use in the method of the invention may incorporate the appropriate transcriptional and translational control sequences, for example enhancer elements, promoter-operator regions, termination stop sequences, mRNA stability sequences, start and stop codons or ribosomal binding sites, linked in frame with the nucleic acid molecules of the invention. These control sequences are provided by way of example only, and are not intended to be limited.

In one embodiment, the level of interaction between Hsp70 and IRE1α may be increased by increasing the level of co-factors of Hsp70. Hsp40 is an example of such a co-factor. The level of cofactor may be increased by any of the methods described above in relation to Hsp70, including administering the protein to the subject, administering a vector encoding the protein to a subject and administering a nuclear localised version of the co-factor, or a vector encoding said co-factor, to a subject.

In another embodiment, the interaction between IRE1α and Hsp70 may be increased by administering an antibody or small molecule to the patient which enhances the level of interaction between IRE1α and Hsp70.

In a further embodiment, the apparent level of interaction been IRE1α and Hsp70 may be increased by administering to the patient an antibody or small molecule which binds to IRE1α and mimics the interaction of Hsp70 with IRE1α.

Within these embodiments, the term antibody includes full chain antibodies and antibody fragments. These include antibody heavy chains, antibody light chains, Fc regions, Fab regions and single chain antibodies. Also contemplated is the use of humanised antibodies, wherein CDR sequences and certain framework residues from a non-human antibody are maintained, and the rest of the antibody residues are substituted for human residues. Methods for the production and humanisation of antibodies are well known in the art.

In one embodiment, the antibody may be a monoclonal antibody.

As it has been determined that Hsp70 and IRE1α interact through the ATPase domain of Hsp70 and the cytosolic C-terminal domain of IRE1α, the antibodies or small molecules for use in these embodiments may be directed against these domains.

In certain embodiments the method of the invention may also comprise inducing cellular stress. The skilled person will understand that various types of cellular stress may be induced, including changes in ER calcium concentration (thapsigargin, calcium ionophore), the disruption of glycosylation (using tunicamycin), the inhibition of ER to Golgi transport (e.g. using brefeldin A), conditions that increase ER protein load (e.g. viral infection), pathological conditions where protein load in the ER increases (e.g. viral infection, accumulation of mutated proteins) and metabolic stress (e.g. exposure to anoxia and ischemia). Within the scope of the invention the method may include inducing cellular stress through inducing one or more of these stressed states. Medical conditions in which cellular stress is a feature require no methods for induction of cellular stress and are preferred conditions in which the present invention may be exploited. Such conditions are listed herein.

The invention also includes a pharmaceutical composition comprising the amino acid sequence of Hsp70 or a vector encoding Hsp70, or the amino acid sequence of IRE1α or a vector encoding IRE1α and a pharmaceutically acceptable carrier, excipient, diluent or buffer.

The team “pharmaceutically acceptable carrier” may include liquids such as water, saline, glycerol, ethanol or auxiliary substances such as wetting or emulsifying agents, pH buffering substances and the like. Excipients may enable the pharmaceutical compositions to be formulated into tablets, pills, capsules, liquids, gels, or syrups to aid intake by the subject. A thorough discussion of pharmaceutically acceptable carriers is available in Remington's Pharmaceutical Sciences [69].

In one embodiment the invention includes the Hsp70, IRE1α, or Hsp40 protein, nucleic acid encoding Hsp70, IRE1α, or Hsp40, or pharmaceutical composition described above for use in the treatment of cancer or a metastatic disease or disorder

In another embodiment the invention includes the use of the Hsp70, IRE1α, or Hsp40 protein, nucleic acid encoding Hsp70, IRE1α, or Hsp40, or pharmaceutical composition described above in the manufacture of a medicament for the treatment of cancer or a metastatic disease or disorder.

As used herein, the term “treatment” is considered to encompass therapy, and can be prophylactic or therapeutic.

The Hsp70, IRE1α, or Hsp40 protein, nucleic acid encoding Hsp70, IRE1α, or Hsp40, or pharmaceutical composition described above may be used for the treatment of disease in any animal. The animal may be a mammal such as a cow, pig, camel, goat, sheep, cat, dog or rabbit. In one embodiment, the mammal may be a human.

The Hsp70, IRE1α, or Hsp40 protein, nucleic acid encoding Hsp70, IRE1α, or Hsp40, or pharmaceutical composition may be formulated as a pill, syrup, injectable solution, cream, ointment, suppository, pessary, spray, eye drop or the Hsp70, IRE1α, or Hsp40 protein, nucleic acid encoding Hsp70, IRE1α, or Hsp40 or pharmaceutical composition may be inserted into or coated onto a medical device useful for the treatment of a patient, such as a stent, stitch, rod or mesh.

The Hsp70, IRE1α, or Hsp40 protein, nucleic acid encoding Hsp70, IRE1α, or Hsp40, or pharmaceutical composition of the invention may be administered to a patient using any one or more of a number of modes of administration which will be known to a person skilled in the art. Such modes of administration may include parenteral injection (e.g. intravenously, subcutaneously, intraperitoneally, intramuscularly, or to the interstitial space of a tissue), or by rectal, oral, vaginal, topical, transdermal, intradermal, intrathecal, intranasal, ocular, aural, pulmonary or other mucosal administration. The precise mode of administration will depend on the disease or condition to be treated.

Treatment Disorders Associated with Increased Apoptosis

A decrease in the level of interaction between Hsp70 and IRE1α will lead to an decrease in the level of activated JNK. This will lead to a decrease in the apoptotic rate. This can be useful for the treatment of a disorders or diseases associated with an increased rate of apoptosis. Therefore, in another aspect the invention provides a method of treating a disease or disorder associated with an increased rate of apoptosis by decreasing the level of interaction between Hsp70 and IRE1α, such that the level of activated JNK rises.

Autoimmune disorders and diseases, allergies or other hypersensitivity disorders, diabetes, cardiac disease, transplant rejection and graft-versus-host disease, and acute and chronic inflammatory diseases are particularly known for having an increased rate of apoptosis.

Within the scope of the invention the autoimmune disorders include but are not limited to achlorhydra autoimmune chronic active hepatitis, Addison's disease, alopecia greata, amyotrophic lateral sclerosis (ALS, Lou Gehrig's Disease), ankylosing spondylitis, anti-GBM nephritis or anti-TBM nephritis, antiphospholipid syndrome, aplastic anemia, arthritis, asthma, atopic allergy, atopic dermatitis, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Balo disease, Behcet's disease, Berger's disease (IgA Nephropathy), bullous pemphigoid, cardiomyopathy, celiac disease, celiac sprue dermatitis, chronic fatigue immune deficiency syndrome (CFIDS), chronic fatigue immune dysfunction syndrome (CFIDS), chronic inflammatory demyelinating polyneuropathy, Churg Strauss syndrome, cicatricial pemphigoid, Cogan's syndrome, cold agglutunin disease, colitis, cranial arteritis, CREST syndrome, Crohn's disease, Cushing's syndrome, Dego's disease, dermatitis, dermatomyositis, dermatomyositis—juvenile, Devic's disease, type 1 diabetes, discoid lupus, Dowling-Dego's disease, Dressler's syndrome, eosinophilic fasciitis, epidermolysis bullosa acquisita, essential mixed cryoglobulinemia, Evan's syndrome, fibromyalgia, fibromyositis, fibrosing alveolitis, gastritis, giant cell artertis, glomerulonephritis, Goodpasture's disease, Grave's disease, Guillian-Barre syndrome, Hashimoto's thyroiditis, hemolytic anemia, Henoch-Schonlein purpura, hepatitis, Hughes syndrome, idiopathic adrenal atrophy, idiopathic pulmonary fibrosis, idiopathic thrombocytopenia purpura (ITP), IgA nephropathy, inflammatory demylinating polyneuropathy, insulin dependent diabetes (Type I), irritable bowel syndrome, juvenile arthritis, Kawasaki's disease, lichen planus, Lou Gehrig's disease, lupoid hepatitis, Lyme disease, Meniere's disease, mixed connective tissue disease, multiple myeloma, multiple sclerosis, myasthenia gravis, myositis, ocular cicatricial pemphigoid, osteoporosis, pars planitis, pemphigus vulgaris, pernicious anemia, polyarteritis nodosa, polychondritis, polyglandular syndromes, polyglandular autoimmune syndromes, polymyalgia rheumatica (PMR), polymyositis, polymyositis and dermatomyositis, primary agammaglobulinemia, primary biliary cirrhois, primary biliary cirrhosis, primary sclerosing cholangitis, psoriasis, Raynaud's phenomenon, Reiter's syndrome, rheumatoid arthritis, sarcoidosis, scleritis, scleroderma, Sjogren's syndrome, sticky blood syndrome, stiff-man syndrome, Still's disease, Sydenham's chorea, systemic lupus erythmatosis (SLE), Takayasu's arteritis, temporal arteritis/giant cell arteritis, ulcerative colitis, uveitis, vasculitis, vitiligo, Wegener's granulomatosis, and Wilson's syndrome.

The allergy or hypersensitivity disorder may be any known allergy or hypersensitivity disorder including type I, type II, type III, or type IV according to the Gell-Coombs classification, and the less commonly defined type V hypersensitivity disorders. Such disorders include but are not limited to atopy, asthma, ertyhroblastosis fetalis, Goodpasture's syndrome, autoimmune hemolytic anemia, serum sickness, Arthus reaction, systemic lupus erythematosus, contact dermatitis, tuberculin skin test, chronic transplant rejection, Graves disease, myasthenia gravis, systemic anaphylaxis, local anaphylaxis, allergic rhinitis, conjunctivitis, gastroenteritis, eczema, blood transfusion reactions, haemolytic disease of the newborn, rheumatoid arthritis, glomerulonephritis, contact dermatitis, atopic dermatitis, tubercular lesions, drug-induced hemolytic anemia, lupus nephritis, aspergillosis, polyarteritis, polymyositis, scleroderma, hypersensitivity pneumonitis, Wegener's granulomastosis, type I diabetes mellitus, urticaria/angioedema, or inflammation of the thyroid. The allergy or hypersensitivity disorder may be associated with infectious diseases including but not limited to tuberculosis, leprosy, blastomycosis, histoplasmosis, toxoplasmosis, leishmaniasis or other infections. Allergies that may be treated include but are not limited to allergic reactions to pollens (e.g. birch tree, ragweed, oil seed rape), food (e.g. nuts, eggs or seafood), drugs (e.g. penicillin or salicylates), insect products (e.g. bee or wasp venom or house dust mites) or animal hair, and man-made products such as latex. Other inflammatory diseases that may be treated include atherosclerosis, Alzheimer's disease, vasculisitis, myositis, encephalitis, reperfusion injury and wound healing, including the inflammatory phase, the process of angiogenesis, fibroplasmia and epithelialisation, and the remodeling phase.

In one embodiment, the level of interaction may be reduced by administering an Hsp70 neutralising agent or an IRE1α neutralising agent to the affected cell.

The level of Hsp70 and/or IRE1α may be reduced by 2-fold, 3-fold, 4-fold, 5-fold, 6-fold, 7-fold, 8-fold, 9-fold, 10-fold, 15-fold, 20-fold or more when compared to a control subject.

In one embodiment the decrease in protein yield may be due to a decreased level of mRNA. The level of Hsp70 and/or IRE1α mRNA may be reduced to a level of 10⁻⁴, 10⁻², 10⁻³, 10⁻⁴, 10⁻⁵, 10⁻⁶ or less mRNA copies per cell relative to the expression level of GAPDH mRNA. The normalisation of the expression level relative to GAPDH is a procedure well known to those skilled in the art.

It will be appreciated that both Hsp70 and IRE1α are involved in complicated pathways within cells, and that both of these proteins have many functions. Therefore, neutralising each of the various functions of either or both of these proteins is likely to have negative effects on a subject. At present it is unclear what the precise effects of a complete neutralisation of either of these proteins would be.

In view of the potential side effects, the skilled person would understand that the “neutralising agent” referred to herein is an agent which is capable of disrupting the interaction between Hsp70 and IRE1α. Such a neutralising agent does not necessarily disrupt or affect any of the other functions and properties of these proteins.

In certain embodiments the neutralising agent may be an Hsp70 neutralising agent or an IRE1α neutralising agent. By this is meant that the neutralising agent is directed to either Hsp70 or IRE1α, respectively. Through this, the neutralising agent is able to disrupt the interaction between Hsp70 and IRE1α.

The inventors have determined that the ATPase domain of Hsp70 and the cytosolic C-terminal region of IRE1α are both required for the interaction between Hsp70 and IRE1α.

It is therefore considered that the ATPase domain of Hsp70 is the point at which Hsp70 interacts with IRE1α. Accordingly, an Hsp70 neutralising agent may be directed to the ATPase domain of Hsp70. As depicted in FIG. 5B, the ATPase domain of Hsp70 is defined as amino acids 1-383 of the amino acid sequence of Hsp72 (SEQ ID NO: 8). Therefore, an Hsp70 neutralising agent may be directed towards, and may bind to one or more of amino acid residues 1-383 of Hsp70.

Alternatively, the ATPase domain of Hsp70 may be required for the interaction with IRE1α because ATP hydrolysis is required for the formation of the interaction.

The cytosolic C-terminal region of IRE1α has been identified as the point at which IRE1α interacts with Hsp70. Accordingly, an IRE neutralising agent may be directed to the cytosolic C-terminal region of IRE1α. As depicted in FIG. 5A, the cytosolic C-terminal region of IRE1α is defined as amino acids 500-967 of the amino acid sequence of IRE1α (SEQ ID NO: 16). Therefore, an IRE1α neutralising agent may be directed towards, and may bind to one or more of amino acid residues 500-967 of IRE1α.

In one embodiment the Hsp70 neutralising agent may be an anti-Hsp70 antibody. The term antibody includes full chain antibodies and antibody fragments. These include antibody heavy chains, antibody light chains, Fc regions, Fab regions and single chain antibodies. Also contemplated is the use of humanised antibodies, wherein CDR sequences and certain framework residues from a non-human antibody are maintained, and the rest of the antibody residues are substituted for human residues. Methods for the production and humanisation of antibodies are well known in the art.

In one embodiment, the antibody may be a monoclonal antibody.

An Hsp70 neutralising antibody for use according to the invention may be directed against one or more residues from the ATPase domain of Hsp70. An IRE1α neutralising antibody may be directed against the cytosolic C-terminal domain of IRE1α.

In other embodiments, the Hsp70 neutralising agent may be a small molecule inhibitor of Hsp70. These include VER-155008 [74], 17-dimethylaminoethylamino-17-demethoxygeldanamycin (17-DMAG) [75], 2-Phenylethylnesulfonamide (PES) [76,77], 17-alkyal-amino-17-demethoxygeldanamycin (17-AAG) [77].

The Hsp70 neutralising agent or the IRE1α neutralising agent may also be a peptide aptamer, DNA aptamer, RNA aptamer or siRNA molecule.

In one embodiment, the invention also provides an Hsp70 neutralising agent or an IRE1α neutralising agent for use in the treatment of autoimmune disorders and diseases, allergies or other hypersensitivity disorders, diabetes, cardiac disease, transplant rejection and graft-versus-host disease, and acute and chronic inflammatory diseases.

The invention also provides the use of an Hsp70 neutralising agent or an IRE1α neutralising agent in the manufacture of a medicament for the treatment of autoimmune disorders and diseases, allergies or other hypersensitivity disorders, diabetes, cardiac disease, transplant rejection and graft-versus-host disease, and acute and chronic inflammatory diseases.

Also included within the scope of the invention is a pharmaceutical composition comprising an Hsp70 neutralising agent or an IRE1α neutralising agent as defined above and a pharmaceutically acceptable carrier, excipient, diluent or buffer. The term “pharmaceutically acceptable carrier” may include liquids such as water, saline, glycerol, ethanol or auxiliary substances such as wetting or emulsifying agents, pH buffering substances and the like. Excipients may enable the pharmaceutical compositions to be formulated into tablets, pills, capsules, liquids, gels, or syrups to aid intake by the subject. A thorough discussion of pharmaceutically acceptable carriers is available in Remington's Pharmaceutical Sciences [69].

In one embodiment, the pharmaceutical composition may include an Hsp70 neutralising agent and an IRE1α neutralising agent. In another embodiment the pharmaceutical composition may include one or more Hsp70 neutralising agents and one or more IRE1α neutralising agents.

The pharmaceutical composition may also include one or more additional therapeutic agents.

As used herein, the term “treatment” is considered to encompass therapy, and can be prophylactic or therapeutic.

The neutralising agents or pharmaceutical compositions described above may be used for the treatment of disease in any animal. The animal may be a mammal such as a cow, pig, camel, goat, sheep, cat, dog or rabbit. In one embodiment, the mammal may be a human.

The neutralising agent or pharmaceutical composition may be formulated as a pill, syrup, injectable solution, cream, ointment, suppository, pessary, spray, eye drop or the neutralising agent or pharmaceutical composition may be inserted into or coated onto a medical device useful for the treatment of a patient, such as a stent, stitch, rod or mesh.

The neutralising agent or pharmaceutical composition of the invention may be administered to a patient using one or more of a number of modes of administration which will be known to a person skilled in the art. Such modes of administration may include parenteral injection (e.g. intravenously, subcutaneously, intraperitoneally, intramuscularly, or to the interstitial space of a tissue), or by rectal, oral, vaginal, topical, transdermal, intradermal, intrathecal, intranasal, ocular, aural, pulmonary or other mucosal administration. The precise mode of administration will depend on the disease or condition to be treated.

### BRIEF DESCRIPTION OF FIGURES

FIG. 1. Hsp72 protects PC12 from apoptosis induced by ER stress. (A) Immunoblotting of total protein from indicated cells was performed using antibodies against Hsp72 and β-actin. (B) The control (Neo) and Hsp72 expressing (Hsp72) PC12 cells were treated (0.25 μM) Tg or (2 μg/ml) Tm for 48 h. Reduction in cell viability was determined by MTT assay. Average and error bars represent mean±SD from three independent experiments performed in triplicates. (C) Cells were treated as in B, and apoptosis was determined with annexin-V/PI staining followed by FACS analysis. Percentages of annexin-V/PI positive cells are shown. Average and error bars represent mean±SD from three independent experiments. (D) Cells were treated as in B, and DEVDase activity was measured as described in materials and methods. Average and error bars represent mean±SD from four independent experiments performed in duplicates. (E) The control (Neo) and Hsp72 expressing (Hsp72) PC12 cells were treated with (0.25 μM) Tg for the indicated time and western blotting of total protein was performed using antibodies against caspase-3 and β-actin. * indicates a statistical significance between Neo and Hsp72 cells; p<0.05. ** indicates a statistical significance between Neo and Hsp72 cells; p<0.005.

FIG. 2. Hsp72 prevents ER stress-induced loss of mitochondrial membrane potential and cytochrome c release. (A) The control (Neo) and Hsp72 expressing (Hsp72) PC12 cells were either untreated (Control) or treated with (0.25 μM) Tg for the indicated time and mitochondrial membrane potential was assessed by TRME staining and flow cytometry. A representative image of three independent experiments is shown. (B) The control (Neo) and Hsp72 expressing (Hsp72) PC12 cells were treated (0.25 μM) Tg for indicated time points. Following treatment cells were incubated with TMRE (100 nM). Mitochondrial membrane potential was monitored by measuring the fluorescence intensity at 582 nm (FL2). Average and error bars represent mean±SD from three independent experiments. (C) The control (Neo) and Hsp72 expressing (Hsp72) PC12 cells were treated with (0.25 μM) Tg for the indicated time. Cytosolic extracts were prepared as described in materials and methods and resolved by SDS-PAGE followed by western blotting using antibodies against Cytochrome c and β-actin.

FIG. 3. ER stress-induced activation of IRE1/XBP1 axis is increased in Hsp72 expressing cells. (A) The control (Neo) and Hsp72 expressing (Hsp72) PC12 cells were either untreated (C) or treated with (0.1 μM) Tg for indicated time points. RT-PCR analysis of total RNA was performed to simultaneously detect both spliced and unspliced XBP1 mRNA and GAPDH. Size of PCR products: unspliced XBP1=289 bp, spliced XBP1=263 bp. The image is presented inverted for greater clarity. (B) In the experiment described in A, XBP-1 mRNA splicing was calculated after densitometric analysis of the XBP-1s PCR products. Average and error bars represent mean±SD from three independent experiments. (C) The control (Neo) and Hsp72 expressing (Hsp72) PC12 cells were treated with (0.25 μM) Tg for the indicated time. Immunoblotting of total protein was performed using antibodies against spliced XBP1 and β-actin. (D) The control (Neo) and Hsp72 expressing (Hsp72) PC12 cells were either untreated (Control) or treated with (0.25 μM) Tg for the indicated time. Immunoblotting of total protein was performed using antibodies against phospho-JNK and β-actin. (E) The control (Neo) and Hsp72 expressing (Hsp72) PC12 cells were treated with (0.25 μM) Tg for the indicated time. Immunoblotting of total protein was performed using antibodies against CHOP, phospho-eIF-2α, total eIF-2α and β-actin. (F) Cells were treated as in A, and the expression a level of indicated genes was quantified by real-time RT-PCR, normalizing against GAPDH. Average and error bars represent mean 1 SD from two independent experiments performed in triplicates. * indicates a statistical significance between Neo and Hsp72 cells; p<0.05. ** indicates a statistical significance between Neo and Hsp72 cells; p<0.005.

FIG. 4. Increased production of spliced XBP1 contributes to cytoprotective function of Hsp72 against ER stress-induced apoptosis. (A) Schematic presentation of wild-type and mutant IRE1α plasmids. (B) PC12 cells were transfected with indicated IRE1α plasmids. 24 h post transfection either untreated (Un) or treated with (0.25 μM) Tg for the 6 h. RT-PCR analysis of total RNA was performed to simultaneously detect both spliced and unspliced XBP1 mRNA and GAPDH. The image is presented inverted for greater clarity. (C) pCMV.SPORT-βGAL was co-transfected with either pcDNA3.1 or IRE1α ΔRNase expression plasmid in control (Neo) or HSP72 expressing (HSP72) PC12 cells. 24 h post transfection cells were either left untreated (UT) or treated with (0.25 μM) Tg for 48 h, (2 μg/ml) Tm for 48 h, (150 nM) staurosporine (STS) for 16 h or (25 μg/ml) etoposide (ETO) for 24 h. The reduction in cell viability was determined by measuring the reduction in β-galactosidase activity after the drug treatments. Average and error bars represent mean±SD from three independent experiments performed in triplicates. ** indicates a statistical significance between Hsp72 and Hsp72 plus IRE ΔRNase cells; p<0.005. (D) Hsp72 expressing PC12 cells were transduced with lentivirus expressing indicated XBP1 targeting shRNA. RT-PCR analysis of total RNA was performed to simultaneously detect unspliced XBP1 mRNA and GAPDH. The image is presented inverted for greater clarity. (E) The control (Neo), Hsp72 expressing (HSP72) and Hsp72 cells expressing indicated shRNAs were either left untreated (UT) or treated with (0.25 μM) Tg, (2 μg/ml) Tm, (150 nM) staurosporine (STS) or (25 μg/ml) etoposide (ETO). The reduction in cell viability was determined by MTT assay. Average and error bars represent mean±SD from three independent experiments performed in triplicates. ** indicates a statistical significance between Hsp72 and Hsp72 plus XBP1 shRNA cells; p<0.005.

FIG. 5. Hsp72 forms a protein complex with IRE1α, and ATPase domain of HSP72 is critical for IRE1α binding. (A) Schematic diagram for IRE1α structure domains and expression constructs. (B) Schematic diagram for Hsp72 structure domains and expression constructs. (C) Hsp72 expressing PC12 cells (Hsp2) were transfected with empty vector (EV) or expression vectors for IRE1α FL-HA or IRE1α ΔC-HA. After 24 h, cells were either left untreated (UT) or treated with (0.25 μM) Tg for 12 h and then the co-precipitation of Hsp72 with IRE1α FL-HA or IRE1α ΔC-HA was evaluated by IP and western blot. (D) PC12 cells expressing the indicated Hsp72 constructs were transfected with IRE1α FL-HA. Lysate from untransfected PC12 cells (UT) was used as a negative control. Co-precipitation of wild-type, ΔATPase and ΔEEVD mutant of Hsp72 with IRE1α FL-HA was evaluated by IP and western blot. (E) HEK 293 cells were transiently transfected with IRE1α FL-HA expression vector or empty vector (EV). After 48 hr, IRE1α FL-HA was immunoprecipitated and its association with endogenous Hsp72 was assessed by western blot. (F) Endogenous Hsp72 was immunoprecipitated from HEK 293 cells transiently transfected with IRE1α FL-HA expression vector or empty vector (EV), and its association with IRE1α was determined by western blot analysis. Input: 5% of the total cell lysate used for IPs.

FIG. 6. The ATPase domain of Hsp72 is necessary for activation of IRE1/XBP1 axis and inhibition of ER stress-induced apoptosis. (A) The control (Neo), wild type HSP72 and ΔATPase Hsp72 expressing PC12 cells were either untreated or treated with (0.25 μM) Tg for 12 h and the expression levels of indicated genes was quantified by real-time RT-PCR, normalizing against GAPDH. Average and error bars represent mean±SD from two independent experiments performed in triplicates. ** indicates a statistical significance between Hsp72 WT and Hsp72 ΔATPase cells; p<0.005. (B) The control (Neo), wild type Hsp72 and ΔATPase Hsp72 expressing PC12 cells were treated (0.25 μM) Tg or (2 μg/ml) Tm for 48 h, and cell viability was determined using MTT assay. Average and error bars represent mean±SD from three independent experiments performed in triplicates. ** indicates a statistical significance between Hsp72 WT and Hsp72 ΔATPase cells; p<0.005. (C) The control (Neo), wild type Hsp72 and ΔATPase Hsp72 expressing PC12 cells were treated as in B, and DEVDase activity was measured as described in materials and methods. Average and error bars represent mean±SD from four independent experiments performed in duplicates. ** indicates a statistical significance between Hsp72 WT and Hsp72 ΔATPase cells; p<0.005.

FIG. 7. Regulation of IRE1α-XBP1 by HSP72 contributes to thermotolerance against ER stress and increased secretion of neurotrophins. (A) PC12 cells were transduced with lentivirus expressing control non-targeting shRNA or XBP1 targeting shRNA. RT-PCR analysis of total RNA was performed to simultaneously detect unspliced XBP1 mRNA and GAPDH. The image is presented inverted for greater clarity. (B) The control (PGIPZ) or XBP1 shRNA expressing (XBP1 shRNA) PC12 cells were heat shocked for 1 h at 42° C. and let to recover for 6 h. Western blot on whole cell lysates were carried out to check the expression of Hsp72 after heat shock with β-actin as loading control (C) Untreated (PGIPZ C, XBP1shRNA C) and heat preconditioned (PGIPZ HS, XBP shRNA HS) control (PGIPZ) and XBP1 shRNA expressing (XBP1 shRNA) PC12 cells were treated with (0.25 μM) Tg for 48 h, (2 μg/ml) Tm for 48 h, (150 nM) staurosporine (STS) for 16 h or (25 μg/ml) etoposide (ETO) for 24 h. The reduction in cell viability was determined by MTT assay. Average and error bars represent mean±SD from three independent experiments performed in triplicates. (D-E) The control (Neo) and Hsp72 expressing (Hsp72) PC12 cells were treated with (0.1 μM) Tg, (0.5 μg/ml) Tm or (50 μM) 6-OHDA for 24 h. Culture supernatant was analysed for NGF and BDNF according to the conditions as described in methods section. Average and error bars represent mean±SD from three independent experiments performed in triplicates.

FIG. 8. Sub-cellular localisation of IRE1 is not affected by Hsp72. Hsp72 and Neo expressing cells were transfected with IRE-GFP. After 24 h of treatment Tg cells were fixed in 3.7% formaldehyde and the coverslips were mounted using Vectashield mounting medium with DAPI (H-1200). Cells were visualised using a Nikon microscope fitted with appropriate filters.

FIG. 9. Overexpression of Hsp72 does not have an effect on the half-life of IRE1. (A) Control (Neo) and Hsp70 expressing (Hsp72) PC12 cells were treated with 10 ng/ml Tg for the indicated time. Immunoblotting of total protein was performed using antibodies against IRE1α and β-actin. (B) In the experiment described in A, band density of IRE1α was calculated by densitometric normalised against β-actin. Average and error bars represent mean±SD from two independent experiments.

FIG. 10. Overexpression of Hsp72 protects PC12 cells from 6-OHDA treatment induced cell death. The control (Neo) and Hsp70 expressing (Hsp72) PC12 cells were treated with 200 mM 6-OHDA for 24 h. Reduction in cell viability was analysed by sub G1 peak. Average and error bars represent mean±SD from three independent experiments performed in triplicate.

FIG. 11. The UPR. A diagrammatic representation of the UPR and its relationship to the apoptotic pathway. The point at which Hsp72 interacts with IRE1α is indicated, and it can be seen that this interaction affects the splicing of XBP1 and increases the levels of XBP1s mRNA and also increases the levels of JNK activity.

FIG. 12. Effect of Hsp72 co-factor Hsp40. Hsp72 expressing PC12 cells (Hsp2) were transfected with empty vector (EV) or expression vectors for IRE1α FL-HA or IRE1α DC-HA. After 24 h, cells were either left untreated (C) or treated with (0.25 mM) Tg for 12 h and then the co-precipitation of Hsp72 with IRE1α FL-HA or IRE1α DC-HA was evaluated by IP and western blot. Input: 5% of the total cell lysate used for IPs.

FIG. 13. HSP72 regulates secretary capacity of UPR primed CHO cells. GFP and Hsp72 expressing CHO cells along with cells induced with thermotolerance were treated with 10 nM Tg for 24 h. Culture supernatant was analysed for PSA by ELISA. The average values of 3 independent experiments were plotted on the graph with the concentration on the Y-axis and treatments on the X-axis.

FIG. 14. Activity and regulation of IRE1α during ER stress. The chaperone GRP78 is bound to the luminal domain of IRE1 during resting conditions. Upon conditions of ER stress, GRP78 dissociates from IRE1 and binds unfolded proteins leading to oligomerisation, trans-autophosphorylation and activation of IRE1. Activated ribonuclease domain of IRE1 splices the mRNA of XBP1 leading to translation of the active transcription factor XBP1s. XBP1s stimulates the transcription of genes involved in re-establishing ER homeostasis and promoting cell survival. The endoribonuclease activity of IRE1 is regulated by BI-1, Bax, Bak and HSP70 that bind to the cytosolic domain of IRE1. IRE1 also splices some other mRNAs leading to their destruction in a mechanism termed regulated IRE1-dependent mRNA decay (RIDD). Upon activation, IRE1 also recruits TRAF2 leading to the down-stream activation of MAP kinases and of transcription factor NFκB, which is thought to promote pro-apoptotic signalling and ER stress-induced cell death.

Various aspects and embodiments of the present invention will now be described in more detail by way of example. It will be appreciated that modification of detail may be made without departing from the scope of the invention.

### EXAMPLES

### Example 1

### Hsp72 Expression Inhibits ER Stress-Induced Apoptosis Upstream of Mitochondria

### Cell Transfection & Culture

The neuroprotective effects of Hsp72 overexpression have been reported in numerous studies during ischemia-like conditions in neuronal cells [15,31,32]. To assess the effect of Hsp72 expression on ER stress-induced apoptosis stable clones of PC12 cells expressing the inducible form of Hsp70 (Hsp72) were generated.

Rat pheochromocytoma PC 12 cells (obtained from ECACC) were cultured in Dulbecco's modified Eagle's medium (DMEM) from Sigma (D6429) supplemented with 10% heat inactivated horse serum, 5% foetal bovine serum and 1% penicillin/streptomycin (Sigma) at 37° C., 5% CO₂ in a humidified incubator. Appropriate number of cells was seeded 24 h prior to treatment.

The plasmid expressing wild type HSP72 was a kind gift from Dr. Tomomi Gotoh, Kumamoto University, Japan [10]. Transfection of cells was carried out using Lipofectamine 2000 (Invitrogen) according to the manufacturer's protocol. Stock solutions of 6-Hydroxydopamine were made freshly in sodium metabisulfite (1 M) prior to experiment. PC12 cells were treated with 200 μM 6-OHDA for 24 h before analysis. All reagents were from Sigma-Aldrich unless otherwise stated.

### Hsp72 Expression

The level of Hsp72 expression in transfected cells was measured by Western blotting. This was performed by washing cells once in ice-cold PBS and lysing cells in whole cell lysis buffer (20 mM HEPES pH 7.5, 350 mM NaCl, 0.5 mM EDTA, 1 mM MgCl₂, 0.1 mM EGTA and 1% NP-40) after the stipulated treatment time and boiling at 95° C. with Laemmli's SDS-PAGE sample buffer for 5 min. Protein concentration was determined by the Bradford method. Equal volumes of sample lysates were run on an SDS polyacrylamide gel. The proteins were transferred onto nitrocellulose membrane and blocked with 5% milk in PBS-0.05% Tween. The membrane was incubated with the primary antibody HSP72 (Stressgen SPA-810) for 2 h at room temperature or overnight at 4° C. The membrane was washed 3 times with PBS-0.05% Tween and further incubated in appropriate horseradish peroxidase-conjugated secondary antibody (Pierce) for 90 min. Signals were detected using West pico chemiluminescent substrate (Pierce).

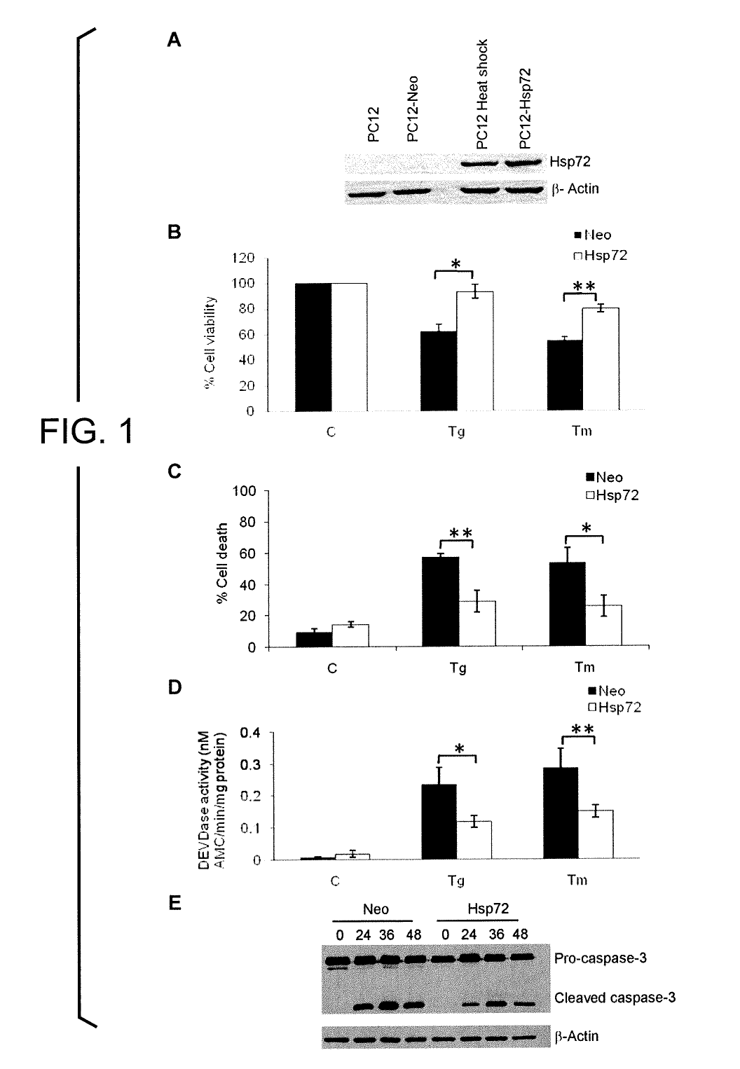

For induction of thermotolerence cells were subjected to 1 h of heat shock at 42° C.±0.5° C. and processed after a 6 h recovery at 37° C. The level of Hsp72 expression in PC12 cells were within the normal physiological range, because ectopic Hsp72 expression is comparable to the level of Hsp72 induced during thermotolerance in PC12 cells (FIG. 1A).

### Cell Viability

To determine the effect of Hsp72 expression on ER stress-induced apoptosis, control (Neo) and Hsp72-expressing (Hsp72) PC12 cells were treated with either 0.25 μM thapsigargin or 1 μg/ml tunicamycin for 48 h.

Cell viability was determined by the MTT assay. After 48 hrs of treatment, 1 mg/ml concentration of MTT ((3-(4,5-dimethylthiazol-2-yl)-2,5-diphenyl tetrazonium bromide) was added to the wells and incubated at 37° C. for 3 hrs. The reaction was stopped with a stop mix containing 20% SDS in 40% dimethyl formamide. The colour intensity was measured at 550 nm and percentage cell viability was calculated using the untreated samples as 100%. It was observed that Hsp72 expression partially protected PC12 cells from ER stress-induced cell death (FIGS. 1B & C).

### Caspase Activation

ER stress-induced caspase activity was measured by analysing DEVDase activity as follows. Cells were harvested and pelleted by centrifugation at 350 g. After washing in PBS, cell pellets were re-suspended in 50 μl of PBS and 25 μl was transferred to duplicate wells of a microtiter plate and snap-frozen in liquid nitrogen. To initiate the reaction, 50 μM of the caspase substrate carbobenzoxy-Asp-Glu-Val-Asp-7-amino-4-methyl-coumarin (DEVD-AMC, Peptide Institute Inc.) in assay buffer (100 mM HEPES, pH 7.5, 10% sucrose, 0.1% CHAPS, 5 mM DTT and 0.0001% Igepal-630, pH 7.25) was added to cell lysates. Liberated free AMC was measured by a Wallac Victor 1420 Multilabel counter (Perkin Elmer Life Sciences) using 355 nm excitation and 460 nm emission wavelengths at 37° C. at 60 sec intervals for 25 cycles. The data was analyzed by linear regression and enzyme activity was expressed as nM of AMC released×min⁻¹×mg⁻¹ total cellular protein.

ER stress-induced caspase activity was found to be significantly reduced in Hsp72 expressing cells as compared with control cells (FIG. 1D). In agreement with reduced caspase activity, Hsp72 expressing cells showed reduced processing of pro-caspase-3 to active caspase-3 (FIG. 1E). The availability of processed caspase-3 was measured using a Western blot assay, as described above using ht eprimary antibody Caspase-3 (Cell Signalling, Cat˜9662). These results suggest that caspase activity is required for ER stress-induced apoptosis and that Hsp72 can inhibit the ability of the cell to activate the caspase cascade.

### Loss of Mitochondrial Membrane Potential (ΔΨm)

The loss of mitochondrial membrane potential (ΔΨm) and mitochondrial outer membrane permeabilization (MOMP) is a hallmark of apoptosis [38,39]. Previous studies have shown that Hsp72 inhibits apoptosis by preventing mitochondrial outer membrane permeabilization and cytochrome c release [10,11].

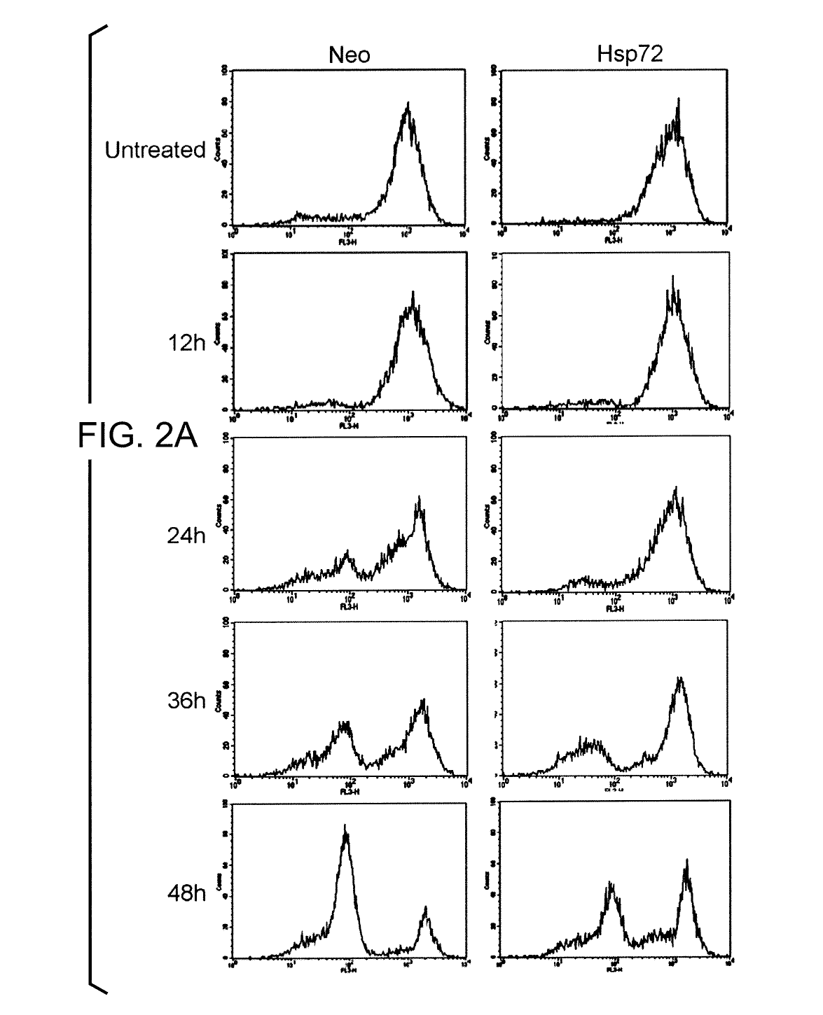

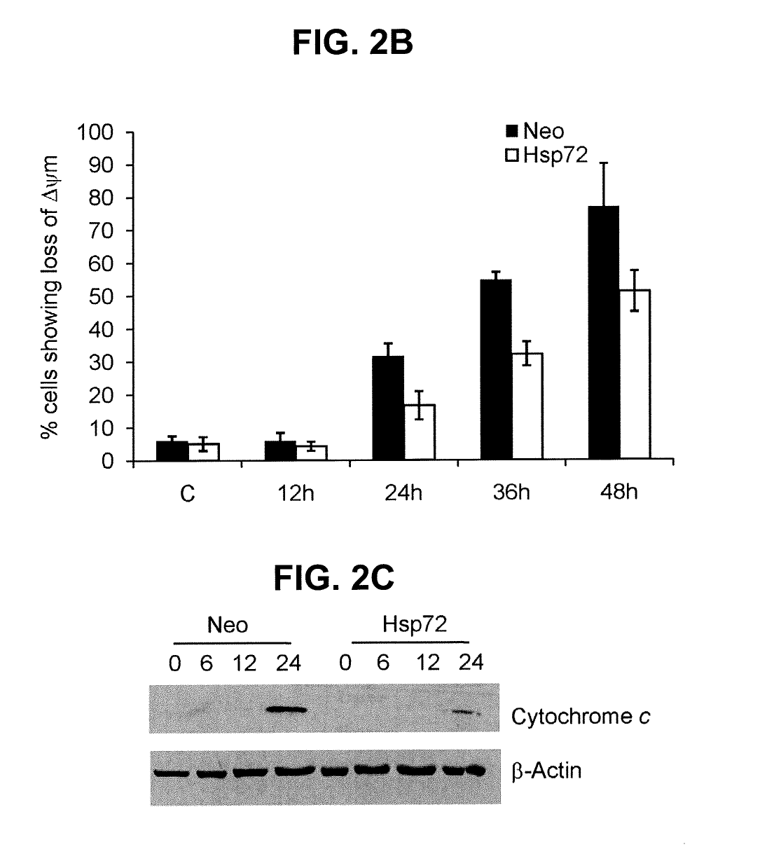

The effect of Hsp72 on the dissipation of ΔΨm and release of cytochrome c to the cytosol upon exposure to ER stress stimuli was evaluated. To quantify ΔΨm, probe tetramethylrhodamine ethyl ester (TMRE, Molecular Probes) [66], a potentiometric fluorescent dye that incorporates into mitochondria in a ΔΨm-dependent manner, was used. Cells were trypsinized and then were either left untreated or treated with 0.25 μM thapsigargin. The cells were then incubated with TMRE for 30 min in the dark and analyzed by flow cytometry using a FACSCalibur instrument. A drop in ΔΨm was observed in Neo cells following thapsigargin treatment (FIG. 2A). The expression of Hsp72 inhibited the loss of ΔΨm (FIG. 2A). At 48 h, loss of ΔΨm was detected in 80-90% of Neo cells treated with thapsigargin or tunicamycin, respectively (FIG. 2B). However, at the same time point, thapsigargin or tunicamycin only induced loss of ΔΨm in 50% of the Hsp72 expressing cells (FIG. 2B).

To further study the involvement of mitochondria in ER stress-induced cell death, the release of cytochrome c into the cytosol was assessed. The cells were washed in ice-cold PBS and lysed using cell lysis and mitochondrial intact buffer (CLAMI) containing 250 mM sucrose, 70 mM KCl dissolved in 1×PBS with 0.5 mM DTT and 2.5 μg/ml Pepstatin and 0.2 g/ml Digitonin. The cells were allowed to swell on ice for 5 min. The cell suspension was centrifuged at 400 g for 5 min and the pellet was removed. The supernatant was transferred to a clean eppendorf tube and the mitochondrial and microsomal fractions were separated by spinning at 20,000 g for 5 min. The cytosolic fraction was removed and prepared for western blot by adding 5× sample buffer.

Western blot analysis of the cytosolic extracts of cells was performed as discussed above using the primary antibody Cytochrome C (BD Pharmingen, Cat˜556433). This analysis showed that exposure of Neo cells to thapsigargin for 24 h triggered release of cytochrome c from mitochondria to the cytoplasm (FIG. 2C). However, at the same time point, the release of cytochrome c induced by thapsigargin was significantly reduced in Hsp72 expressing cells (FIG. 2C). These results suggest that Hsp72 may be acting upstream of MOMP to inhibit ER stress-induced apoptosis.

### Example 2

### Hsp72 Expression Enhances XBP1 mRNA Splicing Under ER Stress Conditions

Activation of the UPR and regulation of protein quality control is essential to restore cellular homeostasis and prevent ER stress-induced apoptosis [18,19]. To investigate the possible regulation of the UPR by Hsp72, the activation of IRE1α/XBP1 and PERK/CHOP axis in control and Hsp72 expressing cells was determined.

XBP1 mRNA Splicing

First the levels of XBP1 mRNA splicing were determined by semi-quantitative RT-PCR. Total RNA was isolated using RNeasy kit (Qiagen) according to the manufacturer's instructions. Reverse transcription (RT) was carried out with 2 μg RNA and Oligo dT (Invitrogen) using 20 U Superscript II Reverse Transcriptase (Invitrogen). The cDNA product was subjected to 25-35 cycles of PCR using the forward primer 5-TTACGAGAGAAAACTCATGGGC-3 and reverse primer 5-GGGTCCAACTTGTCCAGAATGC-3 specific for Rat XBP-1. GAPDH (forward: ACCACAGTCCATGCCATC; reverse: TCCACCACCTGTTGCTG) was used as an endogenous control. For real-time PCR experiments, cDNA products were mixed with 2× TaqMan master mixes and 20× TaqMan Gene Expression Assays (Applied Biosystems) and subjected to 40 cycles of PCR in StepOnePlus instrument (Applied Biosystems). Relative expression was evaluated with ΔΔC_{T} method.

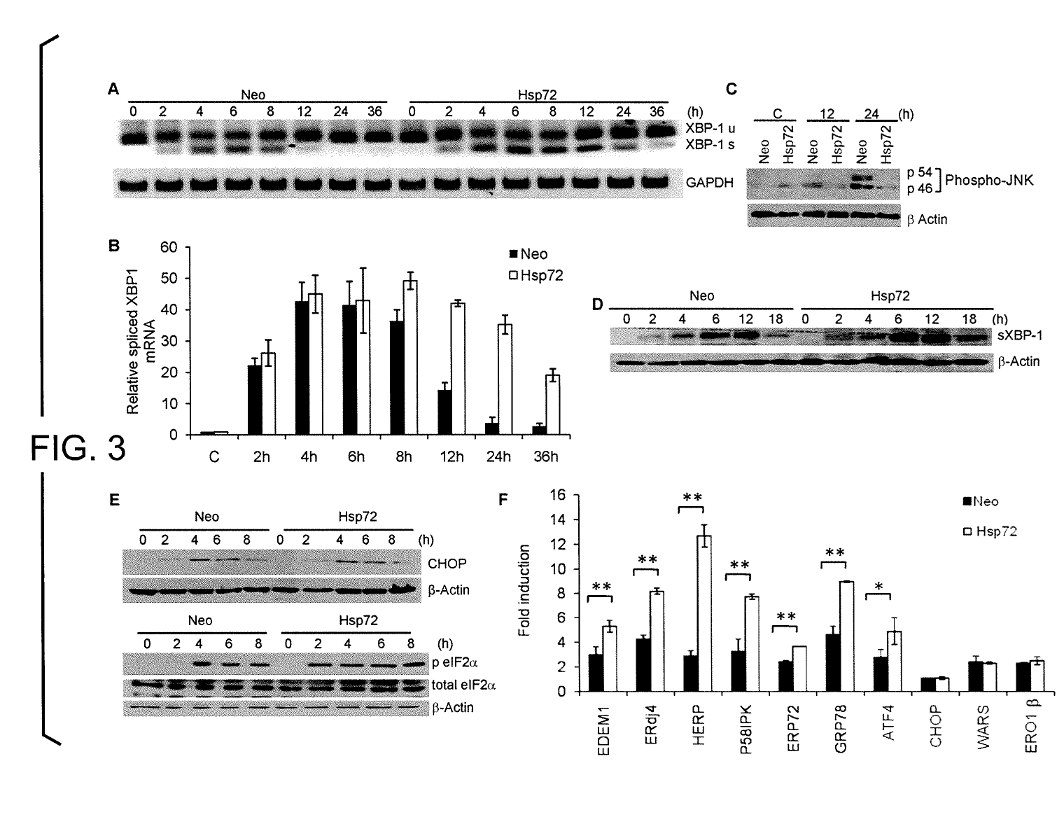

Next, production of spliced XBP1 protein was determined by Western blotting as discussed above using the primary antibody XBP1 (Santa Cruz Biotechnology, Inc, Cat# sc-7160). Notably, upon treatment with thapsigargin Hsp72 cells displayed increased levels of the spliced XBP1 mRNA as compared to Neo cells, observing a sustained signalling over time and late inactivation (FIG. 3A). In agreement with the increased XBP1 mRNA splicing observed above, enhanced expression of XBP1s protein was observed in Hsp72 cells undergoing ER stress when compared with Neo cells (FIG. 3B).

### JNK Activation

Since JNK activation is also induced downstream of IRE1α activation, the effect of Hsp72 on JNK activation during ER stress signalling was determined. Activation of JNK was detected by western blotting as discussed above using the primary antibody JNK (Cell Signalling Cat#92555). ER stress-induced JNK activation was reduced in Hsp72 cells as compared to Neo cells (FIG. 3C).

### Activation of the PERK/CHOP Axis

Activation of the PERK/CHOP axis, a parallel pathway activated by ER stress, was also examined by measuring phosphorylation of eIF-2α, a direct target of PERK, and expression of CHOP. The levels of phosphorylated eIF-2α, and the levels of CHOP were determined by Western blotting as described above, using the primary antibody phosphorylated eIF-2α (Cell Signalling Cat#3597) and the primary antibody CHOP (Santa Cruz Biotechnology, Inc, Cat# sc-973), respectively. The induction of ER stress-induced phosphorylation of eIF-2α and induction of CHOP was not significantly different in Hsp72 cells as compared to Neo cells, although Hsp72 cells showed slightly earlier kinetics in eIF-2α phosphorylation (FIG. 3D). These results indicate that in conditions of ER stress, cellular adaptation is mediated by the transcriptional modulation of the expression of a cohort of UPR target genes. The IRE1α/XBP1 arm of the UPR specifically mediates the induction of specific target genes such as EDEM1, ERdj4, and P58^{1PK}([21,40]. Analysis of gene expression profiles by quantitative RT-PCR, performed as discussed above, revealed that induction of EDEM1, ERdj4, HERP, P58^{1PK} and Grp78 was significantly enhanced in Hsp72 cells as compared to Neo cells (FIG. 3E). Taken together, these observations suggest that Hsp72 specifically regulates ER stress signalling through the modulation of the IRE1α/XBP1 axis of the UPR.

### Example 3

### Increased XBP1s Protein is Required for Enhanced Cell Survival Induced by Hsp72 Under ER Stress Conditions

Recently it has been shown that experimental prolonging of IRE1α signalling independent of ER stress can promote cell adaptation to protein folding stress and survival [9,41]. Here it has been shown that the ability of Hsp72 to inhibit ER stress-induced apoptosis correlates with enhanced production of XBP1s.

### Preparation of a Dominant Negative IRE1α Mutant

To determine the role of XBP1s in the cytoprotective effects of Hsp72 a dominant negative mutant of IRE1α was used to compromise the production of XBP1s and evaluate its effect on the protection mediated by Hsp72.

Stable sub-clones of PC12-Hsp72 with reduced levels of XBP1 were generated by targeting XBP1 mRNA with shRNA using the lentiviral expression vector psiHIV-U6 (GeneCopoeia). The plasmids containing shRNAs targeting rat XBP-1 were obtained from GeneCopoeia, Rockville, USA (RSH045024-HIV U6). Transfection and cell culture were carried out as descried above. The targeting sequences identified for rat XBP1 were XBP1 shRNA1: 5-actgcgcgagatagaaaga-3; XBP1 shRNA2: 5-gttgcctatcagattctg-3; XBP1 shRNA3: 5-gagagccaaactaatgtgg-3 and XBP1 shRNA4: 5-ctgaggtcttcaaaggtat-3.

XBP1 mRNA Splicing

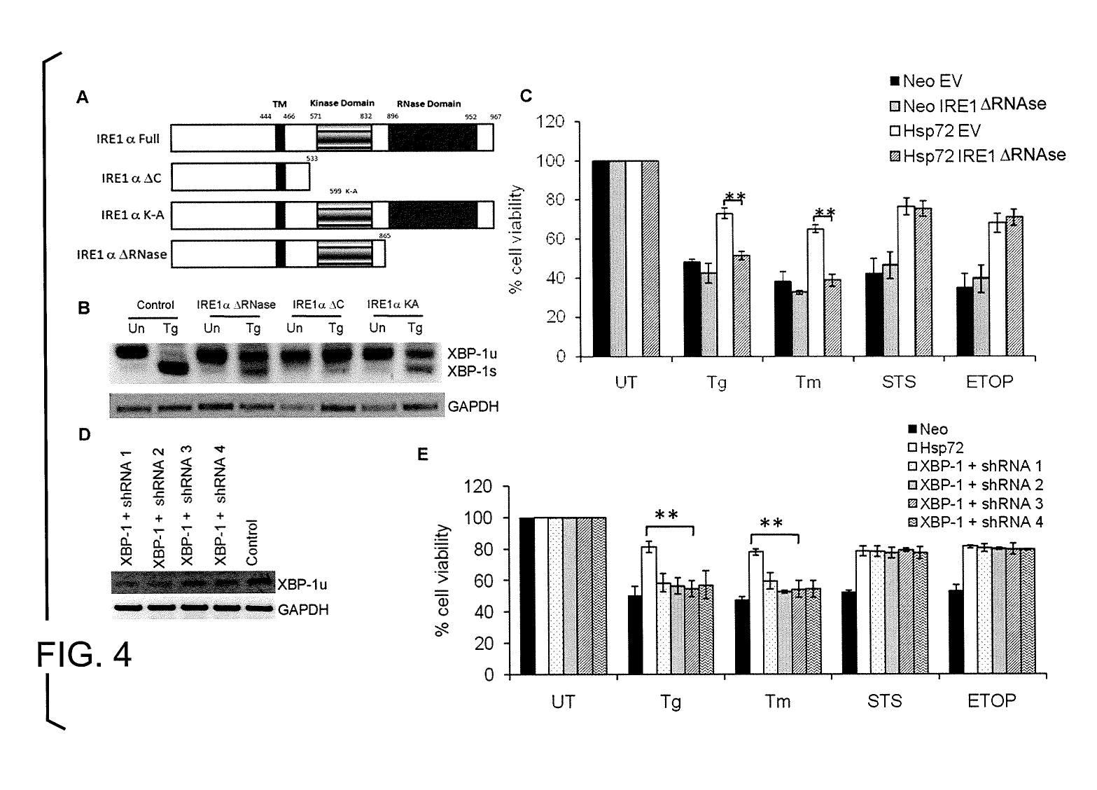

Expression vectors for various mutants of IRE1α (IRE1α KA, IRE1α ΔC and IRE1α ΔRNase) (FIG. 4A) were transfected into PC 12 cells, and the levels of XBP1 mRNA splicing were examined upon ER stress. The plasmids expressing IRE1α KA, IRE1α ΔC and IRE1α ΔRNase were kind gifts from Dr Kazunori Imaizumi, University of Miyazaki, Japan [64]. Transfection and cell culture were carried out as descried above.

The three mutants of IRE1α reduced ER stress-induced splicing of XBP1 as compared to control pcDNA transfected cells (FIG. 4B). Further experiments were performed with the IRE1α ΔRNase because IRE1α KA or IRE1α ΔC mutants may alter the downstream events mediated by the kinase domain of IRE1α in addition to abrogating its endoribonuclease activity. The IRE1α ΔRNase mutant was co-transfected with β-galactosidase plasmid into Neo and Hsp72 cells. Transfection and cell culture were carried out as descried above. After co-transfection of a reporter gene (β-galactosidase), reduction in reporter enzyme activity has been used to determine whether a gene has a detrimental effect on cell survival [42]. The effect of IRE1α ΔRNase on cell viability was determined by measuring β-galactosidase activity after treatment with thapsigargin, tunicamycin, staurosporine and etoposide [42]. Etoposide and staurosporine were used show that expression of IRE1α ΔRNase does not sensitize the cells to other apoptosis inducing agents. The IRE1α ΔRNase mutant specifically attenuated the protective effect of Hsp72 on ER stress-induced apoptosis (FIG. 4C). As control, the non-ER stress agents such as etoposide and staurosporine were used, observing no effects on cells survival after the expression of IRE1α ΔRNase construct.

To further confirm the role of increased XBP1s protein in the cytoprotective effects of Hsp72, XBP1s levels were knocked down by introducing XBP1 targeted shRNAs into Hsp72 cells, as described above, and assessing their effects on cell survival. It was found that all four shRNAs were able to silence XBP1s expression to varying degrees (FIG. 4D). Notably, the protective effect of HSP72 during ER stress-induced apoptosis was abrogated in four independent sub-clones of HSP72 cells expressing XBP1 targeted shRNAs (FIG. 4E). These results suggest that all four XBP1 targeting shRNAs are able to nullify the effect HSP72 overexpression on ER stress-induced production of XBP1s.

The knockdown of XBP1 did not alter the cytoprotective effects of HSP72 on staurosporine- or etoposide-induced apoptosis (FIG. 4E). Collectively, these results suggest that Hsp72 enhances survival under ER stress conditions by upregulation the adaptive responses initiated by the of IRE1α/XBP1 branch of the UPR.

### Example 4

### Hsp72 Forms a Protein Complex with IRE1α

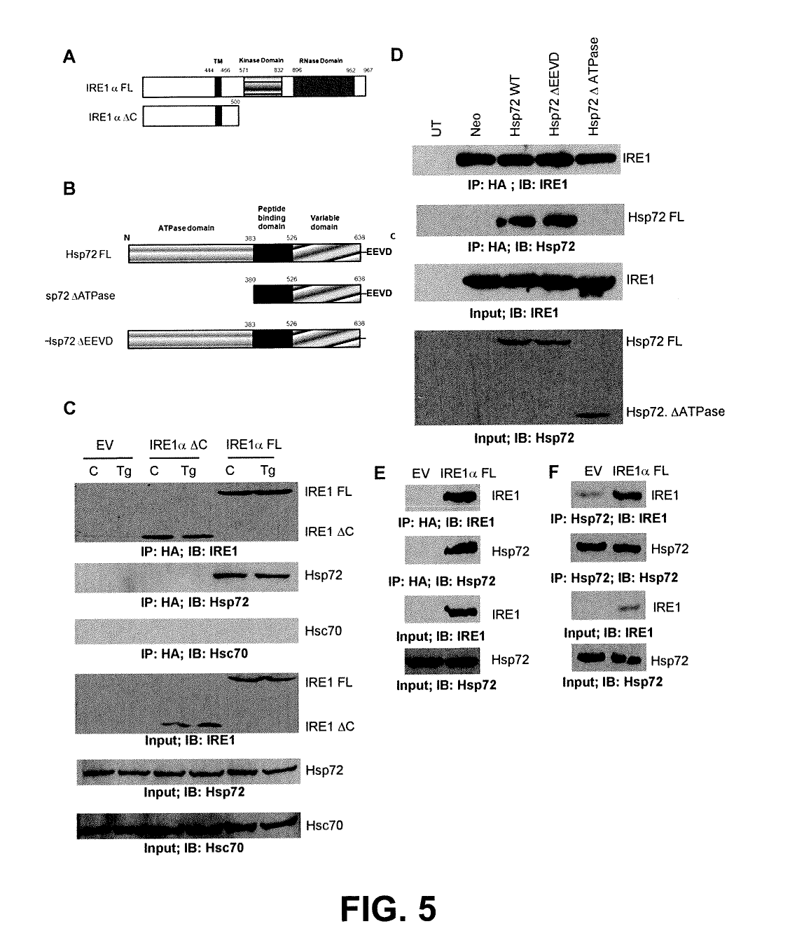

To determine the mechanism by which Hsp72 regulates IRE1α activity, the physical interaction between Hsp72 and IRE1α was investigated. For this purpose Hsp72 expressing PC12 were transfected with wild-type IRE1α FL-HA or IRE1α ΔC-HA (FIG. 5A). Expression plasmids for wild type IRE1α-HA or IRE1α ΔC-HA were as reported previously [28].

### Interaction Between Hsp72 and IRE1α

Interaction of Hsp72-IRE1α was determined by co-immunoprecipitation assays. Immunoprecipitation of HA-tagged wild-type IRE1α or IRE1α ΔC were performed using Pierce Profound mammalian HA tagged IP/Co-IP kit (23615). Briefly, cell lysates were incubated with HA-agarose slurry in IP column overnight. Agarose beads were washed twice with TBS containing 0.05% Tween. Protein complexes were extracted by boiling the beads with 2× lane marker buffer and analysed by western blotting as described above using the primary antibody IRE1α (Cell Signalling Cat#3294S). For Immunoprecipitation of Hsp72, cleared protein extracts were incubated with anti-HSP72 polyclonal antibody (Stressgen SPA-811) overnight at 4° C., followed by 100 μl of a 12% suspension of protein A-Sepharose for 1 h at 4° C. and then washed three times with TBS-0.05% Tween. Protein complexes were eluted by boiling in 2× lane marker buffer and analysed by western blotting as described above using the primary antibody Hsp72 (Stressgen SPA-810).

The Hsp72-IRE1α complex was detected in the absence of ER stress and required the cytosolic C-terminal region of IRE1α, which encodes the kinase and endoribonuclease domains (FIG. 5C). Further, the interaction of Hsp72 with IRE1α was not altered in cells undergoing ER stress triggered by thapsigargin treatment (FIG. 5C). Under similar conditions Hsc70, the constitutive form of Hsp72 did not interact with the cytoplasmic C-terminal region of IRE1α (FIG. 5C).

### Determination of Interacting Domains

Hsp72 consists of three structural motifs: an N-terminal ATPase domain, a C-terminal substrate binding domain and a C-terminal sequence EEVD. Hsp72 function requires coordinated action of all three domains. To map the critical domain of Hsp72 required for IRE1α binding, wild-type IRE1α FL-HA was transfected into PC 12 cells expressing wild-type Hsp72, ΔATPase-Hsp72 or A EEVD-Hsp72 (FIG. 5B). Plasmids expressing wild type HSP72, ΔATPase-HSP72 or A EEVD-HSP72 were kind gifts from Dr. Tomomi Gotoh, Kumamoto University, Japan [10]. The association of IRE1α with HSP72 was determined by co-immunoprecipitation assays, as described above.

Wild-type Hsp72 and ΔEEVD-Hsp72 associated with IRE1α. However, ΔATPase-Hsp72 failed to interact with IRE1α, demonstrating that the ATPase domain of Hsp72 is necessary for interaction of Hsp72 with IRE1α (FIG. 5D). Further the interaction between ectopically expressed wild-type IRE1α FL-HA and endogenous Hsp72 in HEK 293 cells was determined (FIG. 5E).

Endogenous Hsp72 was immunoprecipitated from HEK 293 cells transfected with wild-type IRE1α FL-HA or empty vector as described above. A physical interaction between endogenous Hsp72 and ectopic as well as endogenous IRE1α was observed (FIG. 5F).

### Endoribonuclease Activity

Based on the results of the immunoprecipitation experiments, the possible effects of Hsp72 on the endoribonuclease activity of IRE1α in vitro were monitored. The activity of recombinant human IRE1αN-HIS was produced as a GST fusion protein and purified using the Prescission Protease cleaved system. IRE1α ΔN was incubated with recombinant HSP70 (Stressgene) in a total volume of 50 μl for 1 hr at 30° C. with 10 μg of total mRNA as substrate (obtained from mouse brain cortex because of minimal basal XBP-1 mRNA splicing levels) in a buffer containing 20 mM HEPES (pH 7.3), 1 mM DTT, 10 mM magnesium acetate, 50 mM potassium acetate, and 2 mM ATP. mRNA was re-extracted with 500 ml of Trizol, and the endoribonuclease activity of IRE1α was monitored by RT-PCR using the XBP-1 mRNA splicing assay that employs a set of primers that closely surround the processing site. Using this method, a decrease in the amount of non-spliced XBP-1 mRNA was observed due to its cleavage by IRE1αN-HIS as previously described [28].

### IRE1α Signalling

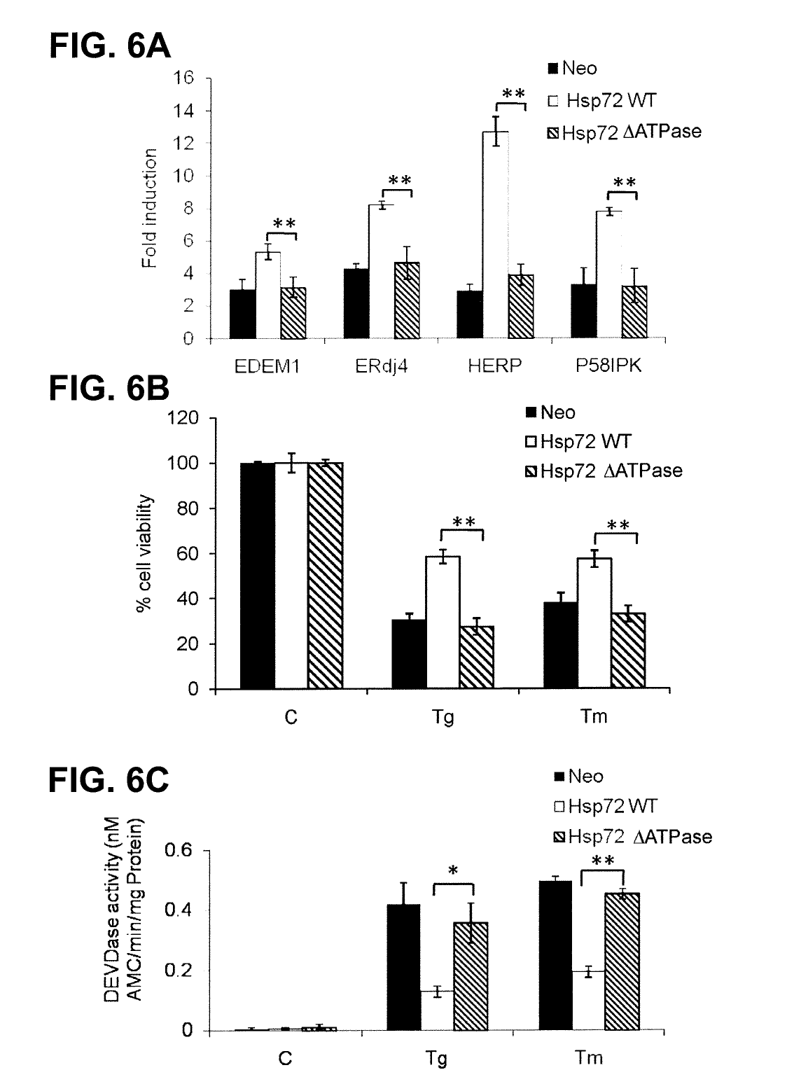

The critical role of the Hsp72 ATPase domain in IRE1α binding led to the determination of its role in ER stress-mediated IRE1α signaling. The induction of EDEM1, ERdj4, HERP, P58^{1PK} and Grp78 in PC12 cells expressing wild-type Hsp72 or ΔATPase-Hsp72 was determined by quantitative RT-PCR analysis, performed as described above. This revealed that induction of EDEM1, ERdj4, HERP, P58^{1PK} (and Grp78 was significantly enhanced only in wild-type HSP72 expressing cells. Notably, induction of EDEM1, ERdj4, HERP, P58^{1PK} and Grp78 in ΔATPase-Hsp72 expressing cells was comparable to Neo cells (FIG. 6A).

### ER Stress Induced Apoptosis Activation

The ER stress-induced apoptosis and caspase activity in PC12 cells expressing wild-type Hsp72 or ΔATPase-Hsp72 was analysed. As shown on FIGS. 6 B & C, wild-type Hsp72 expressing cells were more resistant to ER stress-induced apoptosis and caspase activation. There was no significant difference in ER stress-induced apoptosis and caspase activation in ΔATPase-Hsp72 and Neo cells (FIGS. 6 B & C). Collectively, these results show that the ability of Hsp72 to bind to IRE1α correlates with increased induction of UPR target genes downstream of IRE1α/XBP1 and protection against ER stress-induced cell death.

### Example 5

### Hsp72 Regulates IRE1α-XBP1 in Physiological Models

Mammalian cells, when exposed to a non-lethal heat shock, have the ability to acquire a transient resistance to subsequent exposures at elevated temperatures, a phenomenon termed thermotolerance. It has previously been shown that mild heat shock preconditioning can induce expression of Hsp72 and protect PC 12 cells against a number of cytotoxic agents [43].

### Acquisition of Thermotolerance

To evaluate the effect of Hsp72 on the IRE1α-XBP1 axis in physiological conditions the acquisition of thermotolerance in control and XBP1 knockdown PC12 cells was determined. For this purpose parental PC12 cells were transduced with control (PGIPZ) and XBP1 targeting shRNA (XBP1 shRNA) expressing lentiviral particles. The plasmids containing shRNAs targeting rat XBP-1 were obtained from GeneCopoeia, Rockville, USA (RSH045024-HIV U6) and transfection was performed as described above.

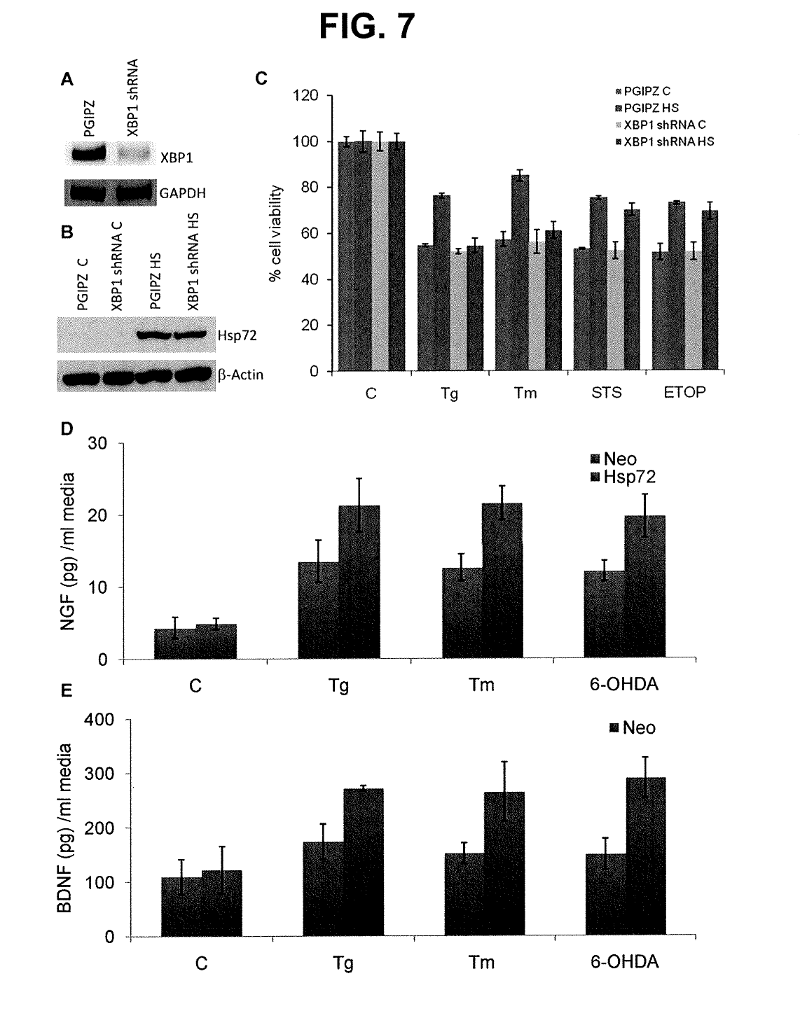

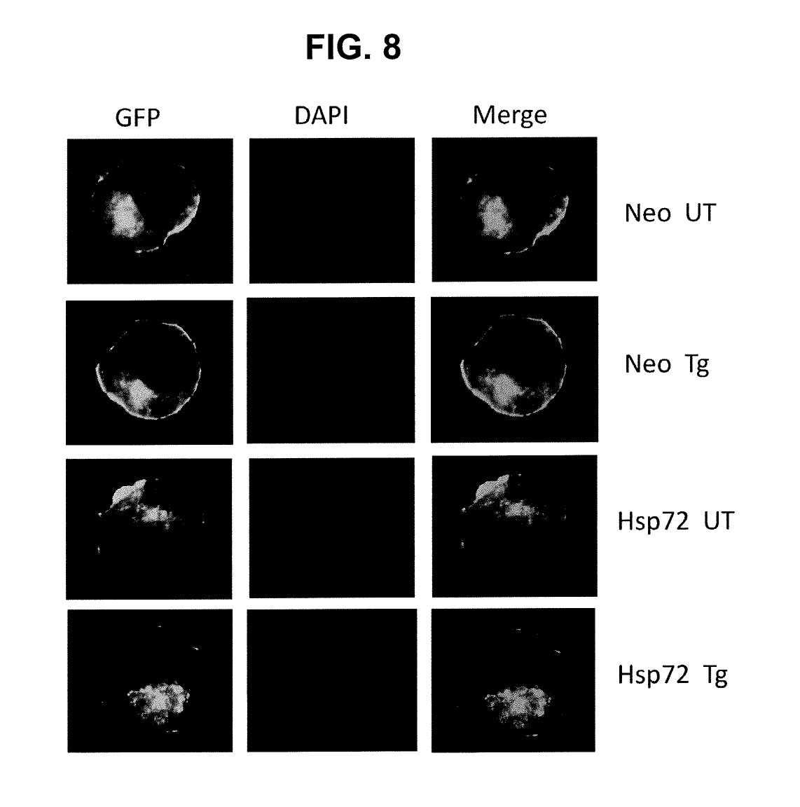

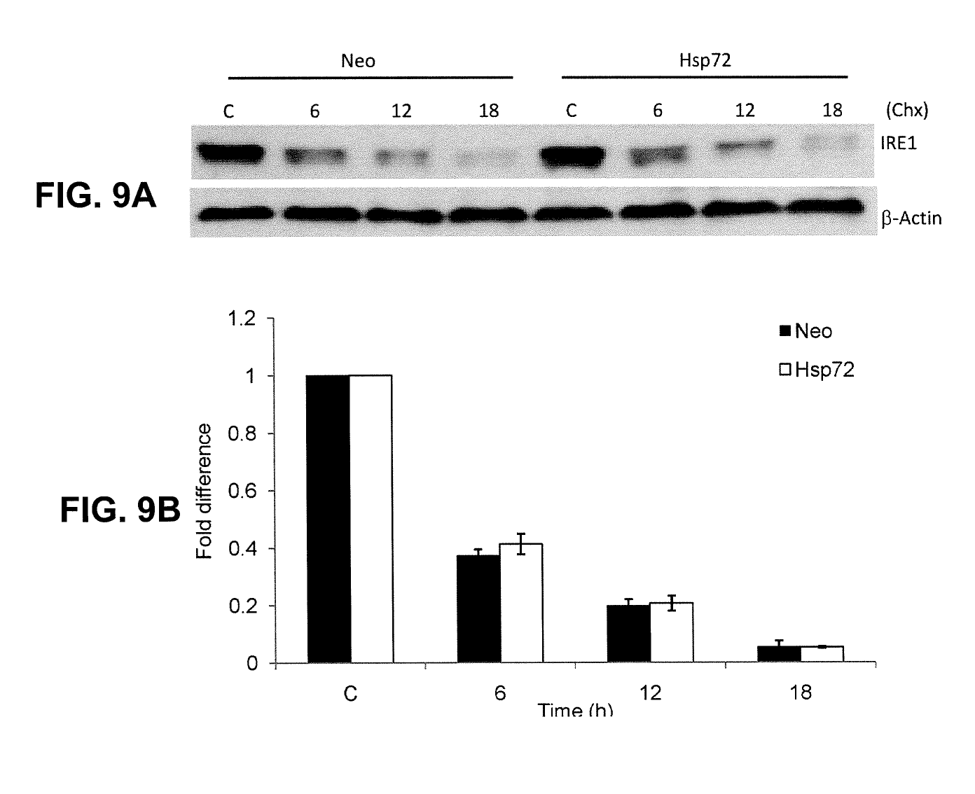

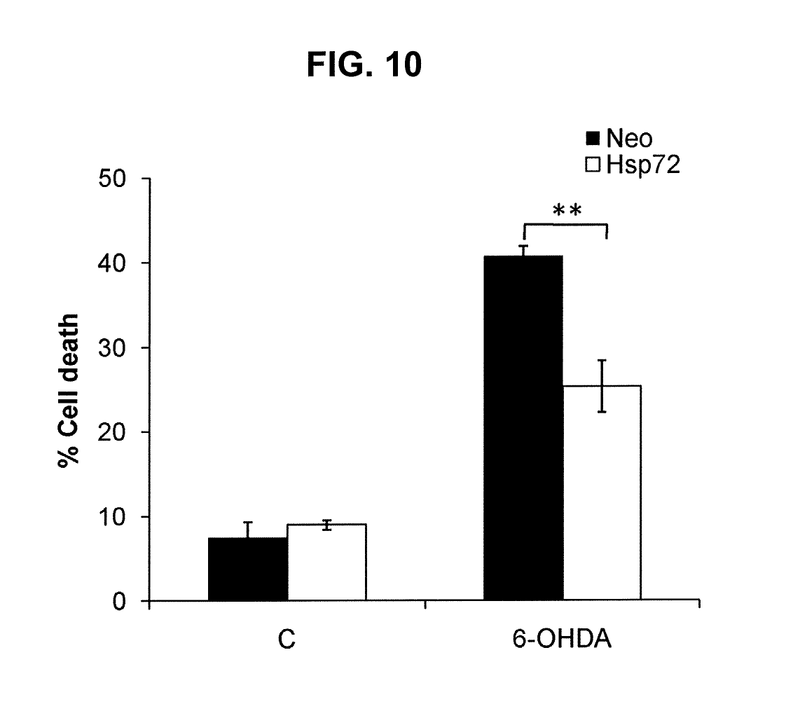

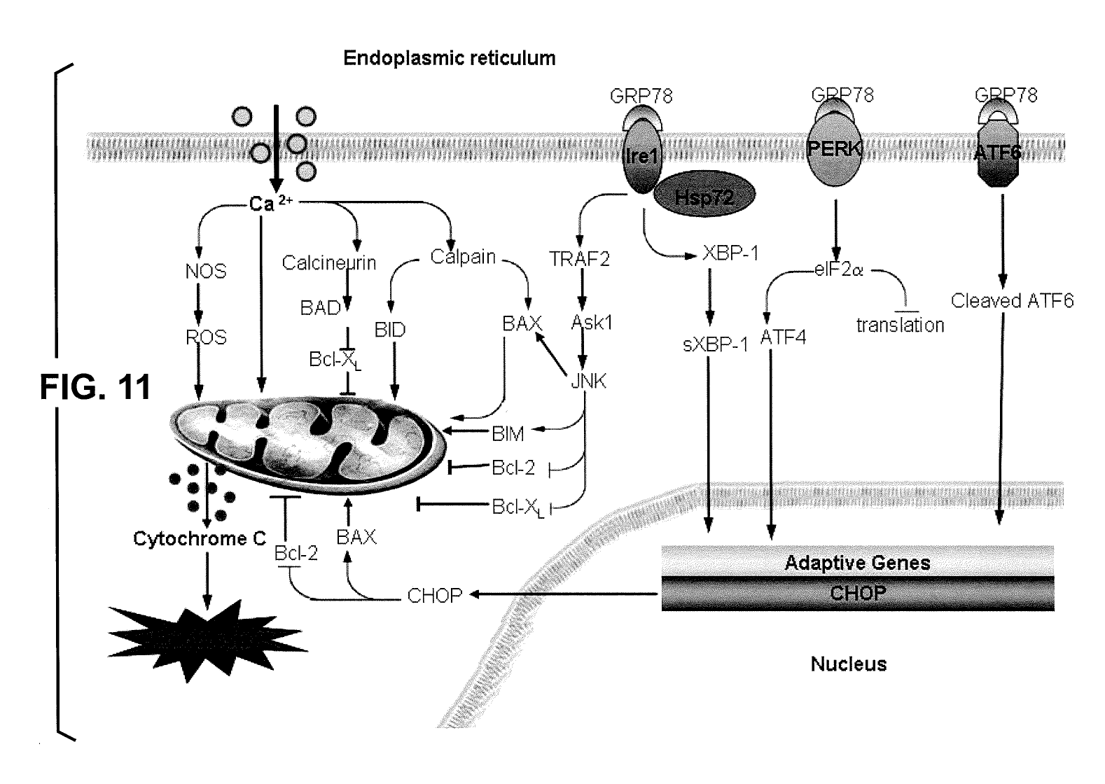

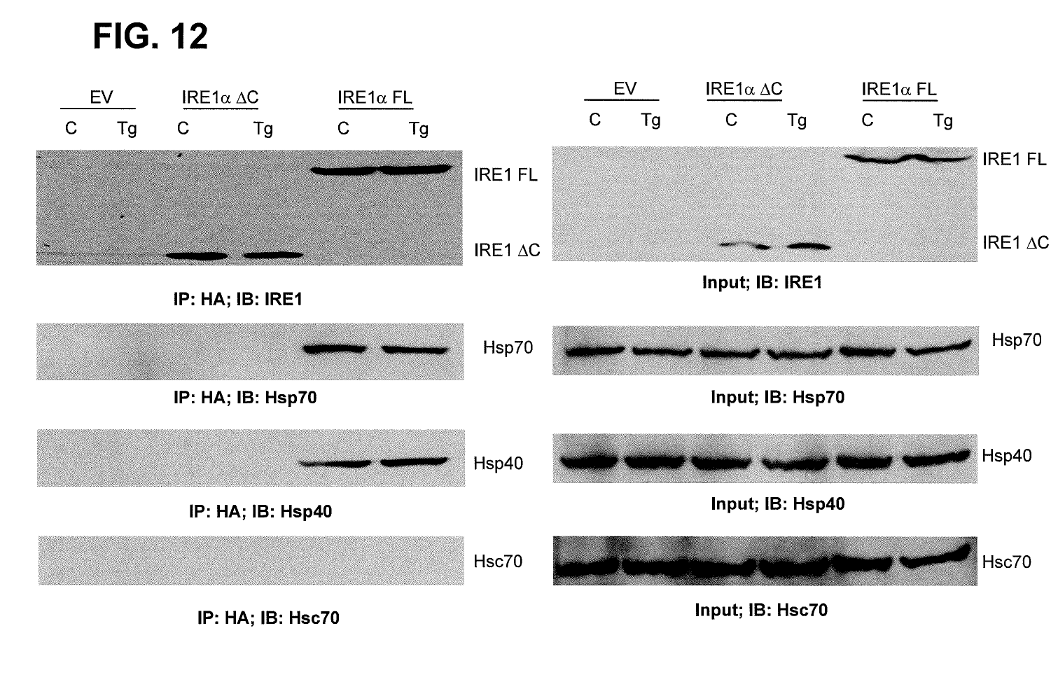

Mild heat shock preconditioning, as described above, induced the expression of Hsp72 in control and XBP1 knockdown PC12 cells to comparable levels (FIGS. 7A & B).

Knockdown of XBP1 specifically abrogated the acquisition of thermotolerance against ER stress-induced apoptosis in PC12 cells (FIG. 7C). The knock down of XBP1 had no effect on acquisition of thermotolerance against etoposide or staurosporine (FIG. 7C). These results suggest an important role for regulation of IRE1α/XBP1 axis by Hsp72 in attainment of thermotolerance induced upon heat preconditioning. More importantly, these data provides a molecular crosstalk between the cytosolic Heat shock response and the UPR.

### Secretion of NGF and BDNF

The main physiological function of the XBP-1 axis of the UPR is to modulate secretory pathway function, enhancing protein secretion [44,45,46]. PC12 is a cell line derived from a pheochromocytoma of the rat adrenal gland and secretes neurotrophins such as nerve growth factor (NGF) and brain-derived neurotrophic factor (BDNF). Therefore, we monitored the secretion of NGF and BDNF in control (Neo) and Hsp72 (Hsp72) expressing cells after exposure to sublethal dose of either thapsigargin (Tg) or tunicamycin (Tm) to modulate ER physiology. Cells were treated with 0.1 μM Tg or 0.5 Tm or 50 μM 6-OHDA for 24 h to induce UPR. Culture media was analysed for NGF or BDNF release using β-NGF (DY 256) or BDNF (DY 248) DuoSet ELISA development kit according to manufacturer's protocol (R&D Systems). The amount of NGF or BDNF released in to the media was calculated using the standard curve generated in parallel with recombinant NGF and BDNF.

In addition, the hydroxylated analogue of dopamine, 6-hydroxy dopamine (6-OHDA) was employed, a commonly used model to mimic Parkinson's disease that also triggers ER stress [47,48]. First the effect of Hsp72 expression on 6-OHDA induced death in PC12 cells was determined. It was found that Hsp72 expressing cells were resistant to 6-OHDA induced death as compared to control (Neo) cells (data not shown). The increased secretion of NGF and BDNF into the cell-culture media of Hsp72 expressing cells after treatment with Tg, Tm and 6-OHDA was observed (FIG. 7D-E). These data indicate that Hsp72 regulates secretion of neurotrophins (NGF and BDNF) by PC 12 cells likely mediated by the modulation of IRE1α/XBP1 function.

All the experiments described were repeated at least twice. Results are expressed as mean±standard deviation. Statistical analyses of the results were done with Student's t test using Graphpad (http://www.graphpad.com).

### Example 6

### Hsp72 Increases PSA Secretion in Hsp72-Transfected CHO Cells

In order to analyse the secretary capacity of CHO-PSA cells after UPR-priming, cells transiently transfected with GFP, cells transiently transfected with Hsp72 and heat shocked cells were treated with 10 nM Tg. All transfections were performed using adenoviral vectors. The cells were incubated at 42° C. for 1 h in the presence of 10 nM Tg and allowed to recover for 6 h at 37° C. before measurements were taken.

To quantify the release of Prostrate-specific antigen (PSA) from CHO cells over-expressing PSA, ELISA was performed. Cells were cultured in DMEM-F12 without supplements and were treated with 10 nM Tg for 24 h. The culture media was used for ELISA carried out using PSA ELISA kit from Abnova (Cat # KA 0208). 50 μl culture media were dispensed to the wells along with 50 μl of zero buffer. Standards of 0, 2, 4, 15, 60 and 120 ng/ml PSA were also dispensed to individual wells. The mixture was incubated in the well for 60 min. After the incubation, media were removed from well and was washed with deionised water 5 times. 100 μA of enzyme conjugate reagent was dispensed to each well and were incubated in RT for a further 60 min. The incubation mixture was removed and the wells were rinsed 5 times. 100 ml of TMB reagent was added to each well and was incubated for 20 min at RT. The reaction was stopped using 100 μl of stop mix and the optical density was read at 450 nm. The quantity of PSA in each ml media was calculated by reference to the curve obtained from the readings of standard solutions.

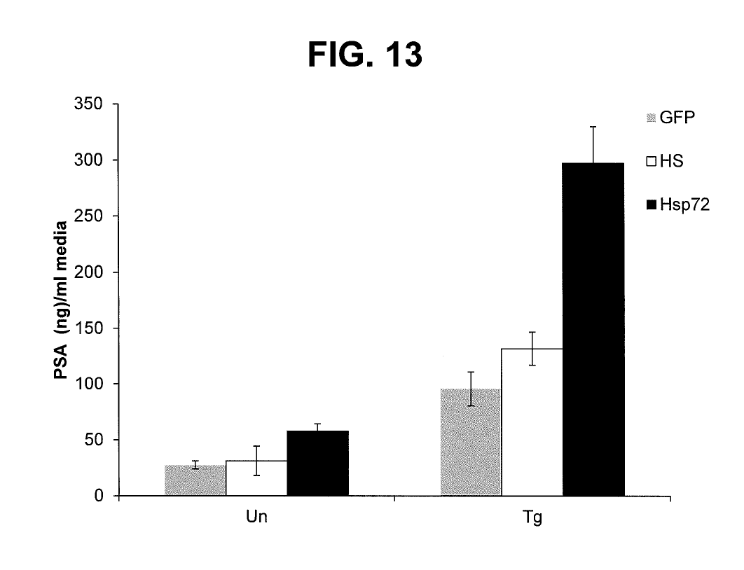

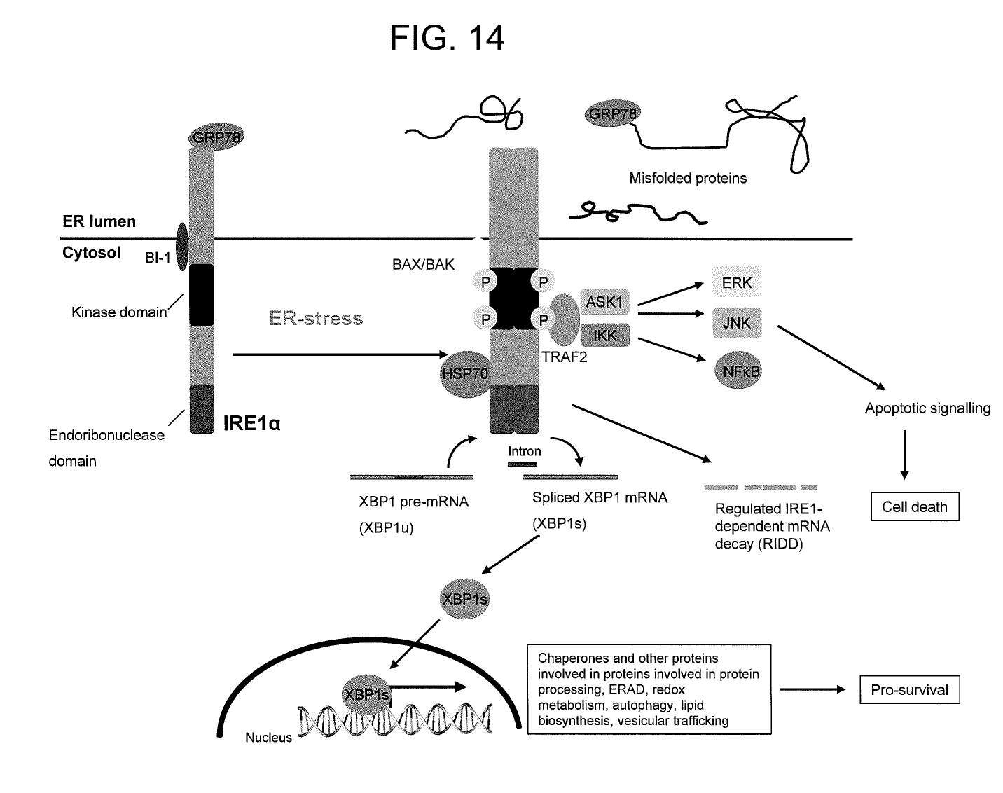

As can be seen in FIG. 13, the presence of the presence of Hsp72 in a cell greatly increases the amount of PSA secretion observed upon administration of Tg. This confirms that Hsp72 regulates secretion of PSA by CHO cells, likely mediated by the modulation of IRE1α/XBP1 function.

### REFERENCES

1. Morimoto R I, Kline M P, Bimston D N, Cotto J J (1997) The heat-shock response: regulation and function of heat-shock proteins and molecular chaperones. Essays Biochem 32: 17-29.
2. Tavaria M, Gabriele T, Kola I, Anderson RL (1996) A hitchhiker's guide to the human Hsp70 family. Cell Stress Chaperones 1: 23-28.
3. Gamido C, Schmitt E, Cande C, Vahsen N, Parcellier A, et al. (2003) HSP27 and HSP70: potentially oncogenic apoptosis inhibitors. Cell Cycle 2: 579-584.
4. Samali A, Orrenius S (1998) Heat shock proteins: regulators of stress response and apoptosis. Cell Stress Chaperones 3: 228-236.
5. Samali A, Cotter TG (1996) Heat shock proteins increase resistance to apoptosis. Exp Cell Res 223: 163-170.
6. Lanneau D, Brunet M, Frisan E, Solary E, Fontenay M, et al. (2008) Heat shock proteins: essential proteins for apoptosis regulation. J Cell Mol Med 12: 743-761.
7. Saleh A, Srinivasula S M, Balkir L, Robbins P D, Alnemri E S (2000) Negative regulation of the Apaf-1 apoptosome by Hsp70. Nat Cell Biol 2: 476-483.
8. Beere H M, Wolf B B, Cain K, Mosser D D, Mahboubi A, et al. (2000) Heat-shock protein 70 inhibits apoptosis by preventing recruitment of procaspase-9 to the Apaf-1 apoptosome. Nat Cell Biol 2: 469-475.
9. Bivik C, Rosdahl I, Ollinger K (2007) Hsp70 protects against UVB induced apoptosis by preventing release of cathepsins and cytochrome c in human melanocytes. Carcinogenesis 28: 537-544.
10. Gotoh T, Terada K, Oyadomari S, Mori M (2004) hsp70-DnaJ chaperone pair prevents nitric oxide- and CHOP-induced apoptosis by inhibiting translocation of Bax to mitochondria. Cell Death Differ 11: 390-402.
11. Stankiewicz A R, Lachapelle G, Foo C P, Radicioni S M, Mosser DD (2005) Hsp70 inhibits heat-induced apoptosis upstream of mitochondria by preventing Bax translocation. J Biol Chem 280: 38729-38739.
12. Gabai V L, Meriin A B, Yaglom J A, Volloch V Z, Sherman M Y (1998) Role of Hsp70 in regulation of stress-kinase JNK: implications in apoptosis and aging. FEBS Lett 438: 1-4.
13. Gurbuxani S, Schmitt E, Cande C, Parcellier A, Hammann A, et al. (2003) Heat shock protein 70 binding inhibits the nuclear import of apoptosis-inducing factor. Oncogene 22: 6669-6678.
14. Kroemer G (2001) Heat shock protein 70 neutralizes apoptosis-inducing factor. ScientificWorldJournal 1: 590-592.
15. Matsumori Y, Hong S M, Aoyama K, Fan Y, Kayama T, et al. (2005) Hsp70 overexpression sequesters AIF and reduces neonatal hypoxic/ischemic brain injury. J Cereb Blood Flow Metab 25: 899-910.
16. Lin J H, Walter P, Yen T S (2008) Endoplasmic reticulum stress in disease pathogenesis. Annu Rev Pathol 3: 399-425.
17. Ron D, Walter P (2007) Signal integration in the endoplasmic reticulum unfolded protein response. Nat Rev Mol Cell Biol 8: 519-529.
18. Schroder M, Kaufman R J (2005) The mammalian unfolded protein response. Annu Rev Biochem 74: 739-789.
19. Szegezdi E, Logue S E, Gorman A M, Samali A (2006) Mediators of endoplasmic reticulum stress-induced apoptosis. EMBO Rep 7: 880-885.
20. Calfon M, Zeng H, Urano F, Till J H, Hubbard S R, et al. (2002) IRE1 couples endoplasmic reticulum load to secretory capacity by processing the XBP-1 mRNA. Nature 415: 92-96.
21. Lee A H, Iwakoshi N N, Glimcher L H (2003) XBP-1 regulates a subset of endoplasmic reticulum resident chaperone genes in the unfolded protein response. Mol Cell Biol 23: 7448-7459.
22. Yamamoto K, Yoshida H, Kokame K, Kaufman R J, Mori K (2004) Differential contributions of ATF6 and XBP1 to the activation of endoplasmic reticulum stress-responsive cis-acting elements ERSE, UPRE and ERSE-II. J Biochem 136: 343-350.
23. Yoshida H, Matsui T, Yamamoto A, Okada T, Mori K (2001) XBP1 mRNA is induced by ATF6 and spliced by IRE1 in response to ER stress to produce a highly active transcription factor. Cell 107: 881-891.
24. Hetz C, Glimcher L (2008) The daily job of night killers: alternative roles of the BCL-2 family in organelle physiology. Trends Cell Biol 18: 38-44.
25. Hetz C, Glimcher LH (2009) Fine-tuning of the unfolded protein response: Assembling the IRE1alpha interactome. Mol Cell 35: 551-561.
26. Hetz C, Bernasconi P, Fisher J, Lee A H, Bassik M C, et al. (2006) Proapoptotic BAX and BAK modulate the unfolded protein response by a direct interaction with IRE1alpha. Science 312: 572-576.
27. Luo D, He Y, Zhang H, Yu L, Chen H, et al. (2008) AIP1 is critical in transducing IRE1-mediated endoplasmic reticulum stress response. J Biol Chem 283: 11905-11912.
28. Lisbona F, Rojas-Rivera D, Thielen P, Zamorano S, Todd D, et al. (2009) BAX inhibitor-1 is a negative regulator of the ER stress sensor IRE1alpha. Mol Cell 33: 679-691.
29. Xu C, Xu W, Palmer A E, Reed J C (2008) BI-1 regulates endoplasmic reticulum Ca2+ homeostasis downstream of Bcl-2 family proteins. J Biol Chem 283: 11477-11484.
30. Gu F, Nguyen D T, Stuible M, Dube N, Tremblay M L, et al. (2004) Protein-tyrosine phosphatase 1B potentiates IRE1 signaling during endoplasmic reticulum stress. J Biol Chem 279: 49689-49693.
31. Yenari M A, Giffard R G, Sapolsky R M, Steinberg G K (1999) The neuroprotective potential of heat shock protein 70 (HSP70). Mol Med Today 5: 525-531.
32. Giffard R G, Yenari M A (2004) Many mechanisms for hsp70 protection from cerebral ischemia. J Neurosurg Anesthesiol 16: 53-61.
33. Xu L, Lee J E, Giffard R G (1999) Overexpression of bcl-2, bcl-XL or hsp70 in murine cortical astrocytes reduces injury of co-cultured neurons. Neurosci Lett 277: 193-197.
34. Plumier J C, Krueger A M, Currie R W, Kontoyiannis D, Kollias G, et al. (1997) Transgenic mice expressing the human inducible Hsp70 have hippocampal neurons resistant to ischemic injury. Cell Stress Chaperones 2: 162-167.
35. Rajdev S, Hara K, Kokubo Y, Mestril R, Dillmann W, et al. (2000) Mice overexpressing rat heat shock protein 70 are protected against cerebral infarction. Ann Neurol 47: 782-791.
36. Kelly S, Zhang Z J, Zhao H, Xu L, Giffard R G, et al. (2002) Gene transfer of HSP72 protects cornu ammonis 1 region of the hippocampus neurons from global ischemia: influence of Bcl-2. Ann Neurol 52: 160-167.
37. Papadopoulos M C, Sun X Y, Cao J, Mivechi N F, Giffard RG (1996) Over-expression of HSP-70 protects astrocytes from combined oxygen-glucose deprivation. Neuroreport 7: 429-432.
38. Zamzami N, Kroemer G (2001) The mitochondrion in apoptosis: how Pandora's box opens. Nat Rev Mol Cell Biol 2: 67-71.
39. Gupta S, Kass G E, Szegezdi E, Joseph B (2009) The mitochondrial death pathway: A promising therapeutic target in Diseases. J Cell Mol. Med.
40. Yoshida H, Matsui T, Hosokawa N, Kaufman R J, Nagata K, et al. (2003) A time-dependent phase shift in the mammalian unfolded protein response. Dev Cell 4: 265-271.
41. Lin J H, Li H, Zhang Y, Ron D, Walter P (2009) Divergent effects of PERK and IRE1 signaling on cell viability. PLoS ONE 4: e4170.
42. Miura M, Yuan J (2000) Transient transfection assay of cell death genes. Methods Enzymol 322: 480-492.
43. Quigney D J, Gorman A M, Samali A (2003) Heat shock protects PC12 cells against MPP+ toxicity. Brain Res 993: 133-139.
44. Iwakoshi N N, Lee A H, Vallabhajosyula P, Otipoby K L, Rajewsky K, et al. (2003) Plasma cell differentiation and the unfolded protein response intersect at the transcription factor XBP-1. Nat Immunol 4: 321-329.
45. Lee A H, Chu G C, Iwakoshi N N, Glimcher L H (2005) XBP-1 is required for biogenesis of cellular secretory machinery of exocrine glands. EMBO J. 24: 4368-4380.
46. Reimold A M, Iwakoshi N N, Manis J, Vallabhajosyula P, Szomolanyi-Tsuda E, et al. (2001) Plasma cell differentiation requires the transcription factor XBP-1. Nature 412: 300-307.
47. Ryu E J, Harding H P, Angelastro J M, Vitolo O V, Ron D, et al. (2002) Endoplasmic reticulum stress and the unfolded protein response in cellular models of Parkinson's disease. J Neurosci 22: 10690-10698.
48. Matus S, Lisbona F, Tones M, Leon C, Thielen P, et al. (2008) The stress rheostat: an interplay between the unfolded protein response (UPR) and autophagy in neurodegeneration. Curr Mol Med 8: 157-172.
49. Kim I, Xu W, Reed JC (2008) Cell death and endoplasmic reticulum stress: disease relevance and therapeutic opportunities. Nat Rev Drug Discov 7: 1013-1030.
50. Steel R, Doherty J P, Buzzard K, Clemons N, Hawkins C J, et al. (2004) Hsp72 inhibits apoptosis upstream of the mitochondria and not through interactions with Apaf-1. J Biol Chem 279: 51490-51499.
51. Sun Y, Ouyang Y B, Xu L, Chow A M, Anderson R, et al. (2006) The carboxyl-terminal domain of inducible Hsp70 protects from ischemic injury in vivo and in vitro. J Cereb Blood Flow Metab 26: 937-950.
52. Ravagnan L, Gurbuxani S, Susin S A, Maisse C, Daugas E, et al. (2001) Heat-shock protein 70 antagonizes apoptosis-inducing factor. Nat Cell Biol 3: 839-843.
53. Volloch V, Gabai V L, Rits S, Sherman M Y (1999) ATPase activity of the heat shock protein hsp72 is dispensable for its effects on dephosphorylation of stress kinase JNK and on heat-induced apoptosis. FEBS Lett 461: 73-76.
54. Holtz W A, O'Malley KL (2003) Parkinsonian mimetics induce aspects of unfolded protein response in death of dopaminergic neurons. J Biol Chem 278: 19367-19377.
55. Gorman A M, Szegezdi E, Quigney D J, Samali A (2005) Hsp27 inhibits 6-hydroxydopamine-induced cytochrome c release and apoptosis in PC12 cells. Biochem Biophys Res Commun 327: 801-810.
56. Bush K T, Goldberg A L, Nigam S K (1997) Proteasome inhibition leads to a heat-shock response, induction of endoplasmic reticulum chaperones, and thermotolerance. Biol Chem 272: 9086-9092.
57. Szokalska A, Makowski M, Nowis D, Wilczynski G M, Kujawa M, et al. (2009) Proteasome inhibition potentiates antitumor effects of photodynamic therapy in mice through induction of endoplasmic reticulum stress and unfolded protein response. Cancer Res 69: 4235-4243.
58. Ciocca D R, Calderwood S K (2005) Heat shock proteins in cancer: diagnostic, prognostic, predictive, and treatment implications. Cell Stress Chaperones 10: 86-103.
59. Koumenis C, Naczki C, Koritzinsky M, Rastani S, Diehl A, et al. (2002) Regulation of protein synthesis by hypoxia via activation of the endoplasmic reticulum kinase PERK and phosphorylation of the translation initiation factor eIF2alpha. Mol Cell Biol 22: 7405-7416.
60. Harris A L (2002) Hypoxia—a key regulatory factor in tumour growth. Nat Rev Cancer 2: 38-47.
61. Lin J H, Li H, Yasumura D, Cohen H R, Zhang C, et al. (2007) IRE1 signaling affects cell fate during the unfolded protein response. Science 318: 944-949.
62. Niwa M, Sidrauski C, Kaufman R J, Walter P (1999) A role for presenilin-1 in nuclear accumulation of Ire1 fragments and induction of the mammalian unfolded protein response. Cell 99: 691-702.
63. Marcu M G, Doyle M, Bertolotti A, Ron D, Hendershot L, et al. (2002) Heat shock protein 90 modulates the unfolded protein response by stabilizing IRE1 alpha. Mol Cell Biol 22: 8506-8513.
64. Ogata M, Hino S, Saito A, Morikawa K, Kondo S, et al. (2006) Autophagy is activated for cell survival after endoplasmic reticulum stress. Mol Cell Biol 26: 9220-9231.
65. Concannon C G, FitzGerald U, Holmberg C I, Szegezdi E, Sistonen L, et al. (2005) CD95-mediated alteration in Hsp70 levels is dependent on protein stabilization. Cell Stress Chaperones 10: 59-65.
66. Samali A, Cai J, Zhivotovsky B, Jones D P, Orrenius S (1999) Presence of a pre-apoptotic complex of pro-caspase-3, Hsp60 and Hsp10 in the mitochondrial fraction of jurkat cells. EMBO J. 18: 2040-2048.
67. Lee Y, Wong K, Tan J, Toh P, Mao Y, Brusic V, Yap M (2009) Overexpression of heat shock proteins (HSPs) in CHO cells for extended culture viability and improved recombinant protein production. Journal of Biotechnology 143: 34-43.
68. Sambrook et al, 1989; Ausubel et al, 1991; Spector, Goldman & Leinwald, 1998.
69. Mack Pub. Co., N.J. 1991.
70. WO05/056596
71. WO2009/077857
72. WO/2009/066174
73. Uemura A, Oku M, Mori K, Yoshida H. (2009) Unconventional splicing of XBP1 mRNA occurs in the cytoplasm during the mammalian unfolded protein response. J Cell Sci. 122(Pt 16):2877-86.
74. Massey A, Williamson D, Browne H, Murray J, Dokurno P, Shaw T, Macias A, Daniels Z, Geoffroy S, Dopson M, Lavan P, Matassova N, Francis G, Graham C, Parsons R, Wang Y, Padfeild A, corner M, Drysdale M, Wood, M (2009) A novel, small molecule inhibitor of Hsc70/Hsp70 potentiates Hsp90 inhibitor induced apoptosis in HCT116 colon carcinoma cells. Cancer Chemother Pharmacol. 1194-1203.
75. Kummar S, Gutierrez M, Gardner E, Chen X, Figg W, Zajac-Kaye M, Chen M, Steinberg S, Muir C, Yancey M, Horneffer Y, Juwara L, Melillo G, Ivy S, Merino M, Neckers L, Steeg P, Conley B, Giaccone G, Doroshow J, Murgo A. (2010) Phase I clinical trials of 17-dimethylamino ethyl amino-17-demethoxygeldanamycin (17-DMAG), a heat shock protein inhibitor, administered twice weekly in patients with advanced malignancies. European Journal of Cancer 46, 340-347.
76. Leu J, Pimkina J, Frank A, Murphy M, George D. (2009) A small molecule inhibitor of inducible heat shock protein 70 (HSP70). Mol. Cell. 36(1) 15-27.
77. Galluzzi L, Giordanetto F, Kroemer G (2009). Targeting Hsp70 for cancer therapy. Molecular cell 36, 176-177.
78. Terada K, Mori M (2000). Human DnaJ homologs dj2 and dj3, and bag-1 are positive chaperones of hsc60. The Journal of Biological Chemistry 275(32), 24728-24734.
79. Urano F, Wang X, Bertolotti A, Zhang Y, Chung P, Harding H P, Ron D. (2000). Coupling of Stress in the ER to Activation of JNK Protein Kinases by Transmembrane Protein Kinase IRE1. Science 287, 664-666.
80. Matsuzawa A, Nishitoh H, Tobiume K, Takeda K, Ichijo H. (2002). Physiological Roles of ASK1-Mediated Signal Transduction in Oxidative Stress- and Endoplasmic Reticulum Stress-Induced Apoptosis: Advanced Findings from ASK1 Knockout Mice. Antioxid. Redox Signal. 4, 415-425.
81. Nishitoh H, Matsuzawa A, Tobiume K, Saegusa K, Takeda K, Inoue K, Hori S, Kakizuka A, Ichijo H. (2002). ASK1 is essential for endoplasmic reticulum stress-induced neuronal cell death triggered by expanded polyglutamine repeats. Genes Dev. 16, 1345-1355.
82. Hollien J, Lin J H, Li H, Stevens N, Walter P, Weissman J S. (2009). Regulated Ire1-dependent decay of messenger RNAs in mammalian cells. J. Cell Biol. 186(3):323-31.
83. Hollien J, Weissman JS. (2006). Decay of endoplasmic reticulum-localized mRNAs during the unfolded protein response. Science. 313(5783):104-7.
84. Han D, Lerner A G, Vande Walle L, Upton J P, Xu W, Hagen A, Backes B J, Oakes S A, Papa F R. (2009). IRE1 alpha kinase activation modes control alternate endoribonuclease outputs to determine divergent cell fates. Cell. 138(3):562-75.
85. Dong B, Niwa M, Walter P, Silverman RH. (2001). Basis for regulated RNA cleavage by functional analysis of RNase L and Ire1p. RNA. 7(3):361-73.
86. Zhang C, Kawauchi J, Adachi M T, Hashimoto Y, Oshiro S, Aso T, Kitajima S. (2001). Activation of JNK and transcriptional repressor ATF3/LRF1 through the IRE1/TRAF2 pathway is implicated in human vascular endothelial cell death by homocysteine. Biochem Biophys Res Commun. 289(3):718-24.

1-75. (canceled)

76. A method of generating a cell for protein production, the method comprising manipulating the cell to alter a level of interaction between Hsp70 and IRE1α.

77. The method of claim 76, wherein the method comprises manipulating the cell to increase the level of interaction between Hsp70 and IRE1α, such that the level of unconventionally-spliced XBP1 (XBP1s) mRNA rises.

78. The method of claim 77, wherein the method comprises increasing a level of expression of at least one of Hsp70 or IRE1α.

79. The method of claim 78, wherein the method comprises transfecting the cell with a plasmid expressing Hsp70 or IRE1α.

80. The method of claim 78, wherein the method comprises inserting a strong promoter that is transcriptionally linked to an endogenous gene encoding Hsp70 or IRE1α.

81. The method of claim 76, wherein the method comprises manipulating the cell to decrease the level of interaction between Hsp70 and IRE1α, such that level of activated JNK or level of activation of JNK decreases.

82. The method of claim 76, wherein the method comprises administering at least one of an Hsp70 neutralizing agent or an IRE1α neutralizing agent to the cell.

83. The method of claim 76, wherein the Hsp70 is Hsp72.

84. The method of claim 76, further comprising isolating a protein of interest from a preparation of the cell.

85. A cell produced according to the method of claim 76.

86. A method of treating a disease, the method comprising modulating an interaction between Hsp70 and IRE1α.

87. The method of claim 86, wherein the method comprises modulating the interaction between Hsp70 and IRE1α such that a level of unconventionally-spliced XBP1 (XBP1s) mRNA is altered.

88. The method of claim 87, wherein the method comprises:
decreasing a level of the interaction between Hsp70 and IRE1α, such that the level of the unconventionally-spliced XBP1 (XBP1s) mRNA is reduced; or
increasing a level of the interaction between Hsp70 and IRE1α, such that the level of the unconventionally-spliced XBP1 mRNA rises.

89. The method of claim 88, wherein the disease is selected from the group consisting of cancer, an autoimmune disease, diabetes and cardiac disease.

90. The method of claim 88, comprising administering at least one of an Hsp70 neutralizing agent or an IRE1α neutralizing agent to an affected cell.

91. The method of claim 88, wherein the Hsp70 is Hsp72.

92. The method of claim 90, wherein the Hsp70 neutralizing agent is an anti-Hsp70 antibody, and wherein the IRE1α neutralizing agent is an anti-IRE1α antibody.

93. The method of claim 92, wherein the anti-Hsp70 antibody is an anti-HSP70 monoclonal antibody and wherein the anti-IRE1αantibody is an anti-IRE1α monoclonal antibody.

94. The method of claim 92, wherein the anti-Hsp70 antibody is directed to an ATPase domain of Hsp70.

95. The method of claim 92, wherein the anti-IRE1α antibody is directed to a cytosolic C-terminal region of IRE1α.

96. The method of claim 88, wherein the method further comprises inducing cellular stress.

97. The method of claim 86, wherein the method comprises modulating the interaction between Hsp70 and IRE1α such that a level of activated JNK or a level of JNK activation is altered.

98. A pharmaceutical composition comprising an Hsp70 neutralizing agent and/or an IRE1α neutralizing agent and a pharmaceutically acceptable carrier or the amino acid sequence of Hsp70 or a plasmid encoding Hsp70, and/or the amino acid sequence of IRE1α or a plasmid encoding IRE1α and a pharmaceutically acceptable carrier.

99. A method of treating a disease comprising administering the pharmaceutical composition of claim 98 to an animal, wherein the disease selected from the group consisting of cancer, an autoimmune disease, diabetes and cardiac disease.
